(19) United States
(12) Patent Application Publication
(10) Pub. No.: US 2009/0221596 A1
(43) Pub. Date: Sep. 3, 2009

(54) Substtituted aryl oximes

(76) Inventors: Iris Escher, Aschenberg (DE); Michael Müller, Düsseldorf (DE); Peter Jeschke, Bergisch Gladbach (DE); Michael Beck, Monheim (DE); Oliver Gaertzen, Köln (DE); Olga Malsam, Rösrath (DE); Peter Lösel, Leverkusen (DE); Ulrich Ebbinghaus-Kintscher, Dortmund (DE); Christian Arnold, Langenfeld (DE); Karl-Josef Haack, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/920,266

(22) PCT Filed: May 6, 2006
(86) PCT No.: PCT/EP2006/004256

§ 371 Date: Jan. 16, 2009

(51) Int. Cl. A01N 33/24; C07C 251/54; C07D 213/63; C07D 241/10; C07C 233/00; A01N 43/40; A01N 37/18; A01N 43/60; A01N 43/78

(52) U.S. Cl. 514/252.1; 564/256; 546/300; 544/336; 564/192; 514/640; 514/351; 514/617; 514/625; 514/365

(57) ABSTRACT

The invention relates to compounds of the formula (I)

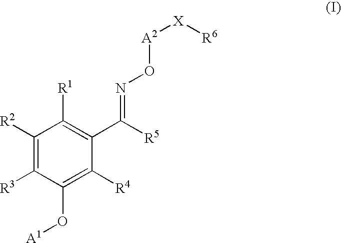

in which
A¹, A², R¹, R², R³, R⁴, R⁵ and X are as defined in the description,
to processes and intermediates for their preparation, and to their use for controlling pests.

The present invention relates to new substituted aryl oximes, to processes for preparing them and to their use as pesticides.

Substituted aryl oximes have already been disclosed as pesticides (cf. WO2003/042147A1).

New substituted aryl oximes have now been found of the general formula (I)

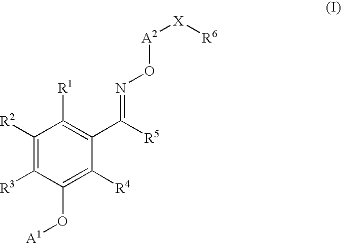

in which
A¹ is one of the moieties —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—C(halogen)₃, —(CH₂)₂—CH(halogen)₂, —CH₂—CH(halogen)-CH(halogen)₂, —CH₂—CH═CClCH₃ and in which
A²-X is a bridging element. Located between X and R⁶, optionally, is the bridging element

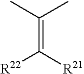

in which the radicals R²¹ and R²² independently of one another are hydrogen, nitro, hydroxyl, amino, cyano, cyanato, thiocyanato, formyl, halogen, in each case optionally cyano-, halogen- or C₁-C₆-alkoxy-substituted alkyl, alkoxy, alkylthio, alkylsulphinyl, alkylsulphonyl, alkylamino, dialkylamino or alkylcarbonylamino having in each case 1 to 4 carbon atoms in the alkyl groups, are C₁-C₄-alkyl-carbonyl, C₁-C₄-alkoxy-carbonyl, C₁-C₄-alkoximinoformyl, C₁-C₄-alkoximino-acetyl, or are C₂-C₄-alkenyl or C₂-C₄-alkynyl.
A²-X is either a direct bond or a straight-chain or branched or cyclic alkanediyl or an alkenediyl having in each case up to 8 carbon atoms and which in each case, optionally in attachment to the carbon chain, contains an oxygen atom, sulphur atom or a moiety selected from SO, SO₂, NH and N(C₁-C₄-alkyl).
A²-X is additionally
   —(C₁-C₄-alkyl)-(CO)—O—, (C₁-C₄-alkyl)-(CO)—(NH)—, —(C₁-C₄-alkyl)-(CO)-[N—(C₁-C₄-alkyl)]-, —(C₁-C₄-alkyl)-(CO)—S—, —(C₁-C₄-alkyl)-O—(CO)—, —(C₁-C₄-alkyl)-(NH)—(CO)—, —(C₁-C₄-alkyl)-[N—(C₁-C₄-alkyl)]-(CO)—, —(C₁-C₄-alkyl)-S—(CO)—, —(C₂-C₈-alkenyl)-(CO)—O—, —(C₂-C₈-alkenyl)-(CO)—(NH)—, —(C₂-C₈-alkenyl)-(CO)-[N—(C₁-C₄-alkyl)]-, —(C₂-C₈-alkenyl)-(CO)—S—, —(C₂-C₈-alkenyl)-O—(CO)—(C₂-C₈-alkenyl)-(NH)—(CO—)—, —(C₁-C₄-alkyl)-[N—(C₁-C₄-alkyl)]-(CO)—, —(C₂-C₈-alkenyl)-S—(CO)—, —(C₁-C₄-alkyl)-[N—(C₁-C₄-alkyl)]-(CO)—, —(C₂-C₈-alkenyl)-S—(CO)—, —(C₁-C₄-alkyl)-(SO₂)—(NH)—, —(C₂-C₈-alkenyl)-(CO)—S—, —(C₂-C₈-alkenyl)-(SO₂)—(NH)—, —(C₁-C₄-alkyl)-O—N═C(R⁹)—, —(C₁-C₈-alkenyl)-O—N═C(R⁹)—,
R⁹ is C₁-C₄-alkyl or is aryl,
A²-X is further a carbocyclic, optionally mono- or polyunsaturated, 5-, 6- or 7-membered ring system or is an optionally mono- or polyunsaturated, 7-10-membered bicyclic ring system, which optionally may be interrupted one or more times by oxygen, sulphur, sulphonyl, sulphoxyl, carbonyl, NO, or by optionally alkyl-substituted nitrogen, and optionally may be substituted one or more times. The attachment of these ring systems to the oxime oxygen is always via carbon. The attachment to the radical R⁶ may be via carbon or a heteroatom (nitrogen or sulphur).
R¹ is hydrogen, nitro, hydroxyl, amino, cyano, fluoro, chloro, bromo, iodo, or is in each case optionally cyano-, fluoro-, chloro-, methoxy-, ethoxy-, n- or isopropoxy-substituted C₁-C₆-alkyl, C₃-C₆-cycloalkyl, C₃-C₆-cycloalkyloxy, C₃-C₆ alkenyl, C₃-C₆ alkenyloxy, C₃-C₆ alkynyl, C₃-C₆ alkynyloxy, C₁-C₆ alkylcarbonyloxy, C₁-C₃ alkylsulphonyl, methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino, dimethylamino, diethylamino, dipropylamino, acetylamino, propionylamino, n- or isobutyroylamino, methoximinomethyl, ethoximinomethyl, methoximinoethyl or ethoximinoethyl,
R² is hydrogen, nitro, hydroxyl, amino, cyano, cyanato, thiocyanato, formyl, halogen, is in each case optionally cyano-, halogen- or C₁-C₆-alkoxy-substituted alkyl, alkoxy, alkylthio, alkylsulphinyl, alkylsulphonyl, alkylamino, dialkylamino or alkylcarbonylamino having in each case 1 to 6 carbon atoms in the alkyl groups, is C₁-C₆-alkyl-carbonyl, C₁-C₆-alkoxy-carbonyl, C₁-C₆-alkoximinoformyl, C₁-C₆-alkoximino-acetyl, or is C₂-C₆-alkenyl or C₂-C₆-alkynyl,
R³ is hydrogen, nitro, hydroxyl, amino, cyano, halogen, is in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁴ is hydrogen, nitro, hydroxyl, amino, cyano, halogen, is in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁵ is hydrogen, straight-chain or branched or cyclic halogenalkanyl or halogenalkenyl having in each case up to 8 carbon atoms, aryl, substituted aryl,
R⁶ is an alkenyl moiety, an alkynyl moiety having in each case 2 to 6 carbon atoms or cyclo-alkenyl moiety having 4 to 6 carbon atoms, which is optionally substituted by at least one substituent from the group nitro, cyano, carboxyl, carbamoyl, hydroxyl, carbonyl (C═O), hydroximino (C═N—OH), C₁-C₆-alkoxy, C₁-C₆-alkoxy-carbonyl, C₁-C₆-alkylamino, di(C₁-C₆-alkyl)amino, C₁-C₆-alkylamino-carbonyl, C₁-C₆-alkoxy-carbonylamino, C₁-C₆-alkoxy-C₁-C₆-alkoxy, C₁-C₆-alkoxyimino, C₃-C₆-alkenyloxy, C₃-C₆-alkynyloxy, C₃-C₆-alkenyloxy-carbonyl, C₃-C₆-alkynyloxy-carbonyl, C₃-C₆-alkenyloxyimino, C₃-C₆-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl.
R⁶ is additionally a moiety -A³-Z, where
   A³ is a single bond or is straight-chain or branched and optionally halogen- or C₃-C₆-cycloalkyl-substituted alkanediyl having 1 to 6 carbon atoms and
   Z is optionally nitro-, hydroxyl-, mercapto-, amino-, formyl-, cyano-, carboxyl-, carbamoyl-, halogen-, C₁-C₆-alkyl-, C₁-C₆-hydroxyalkyl-, C₁-C₆-haloalkyl-, C₁-C₆-alkyl-carbonyl-, C₁-C₆-haloalkyl-carbonyl-, C₁-C₆-alkoxy-, C₁-C₆-hydroxyalkoxy-, C₁-C₆-haloalkoxy-, C₁-C₆-alkoxy-carbonyl-, C₁-C₆-haloalkoxy-carbonyl-, C₁-C₆-alkoxy-C₁-C₆-alkyl-, C₁-C₆-haloalkoxy-C₁-C₆-alkyl-, C₁-C₂-alkylenedioxy-, C₁-C₂-haloalkylenedioxy-, C₁-C₆-alkoxyimino-C₁-C₆-alkyl-, C₂-C₆-alkenyl-, C₂-C₆-alkenyl-carbonyl-, C₂-C₆-haloalkenyl-, C₂-C₆-haloalkenyl-carbonyl-, C₂-C₆-alkenyloxy-C₁-C₆-alkyl-, C₂-C₆-haloalkenyloxy-C₁-C₆-alkyl-, C₂-C₆-alkynyl-, C₂-C₆-haloalkynyl-, C₂-C₆-alkenyloxy-, C₂-C₆-alkenyloxy-carbonyl-, C₂-C₆-haloalkenyloxy-, C₂-C₆-haloalkenyloxy-carbonyl-, C₂-C₆-alkynyloxy-, C₂-C₆-alkynyloxy-carbonyl-, C₂-C₆-haloalkynyloxy-, C₂-C₆-haloalkynyloxy-carbonyl-, C₂-C₆-alkynyloxy-C₁-C₆-alkyl-, C₂-C₆-haloalkynyloxy-C₁-C₆-alkyl-, C₃-C₈-cycloalkyl-, C₃-C₈-cycloalkyl-carbonyl-, C₃-C₈-cycloalkyl-C₁-C₆-alkyl-, C₃-C₈-cycloalkyl-C₁-C₆-alkyl-carbonyl-, C₃-C₈-cycloalkyloxy-, C₃-C₈-cycloalkyloxy-carbonyl-, C₃-C₈-cycloalkyl-C₁-C₆-alkoxy-, C₃-C₈-cycloalkyl-C₁-C₆-alkoxy-carbonyl-, C₃-C₈-cycloalkyloxy-C₁-C₆-alkyl-, C₃-C₈-cycloalkyloxy-C₁-C₆-alkoxy-, C₃-C₈-cycloalkyl-C₁-C₆-alkoxy-C₁-C₆-alkyl-, C₁-C₆-alkyl-carbonyl-C₁-C₆-alkyl-, C₁-C₆-alkoxy-carbonyl-C₁-C₆-alkyl-, C₁-C₆-alkylthio-, C₁-C₆-haloalkylthio-, C₁-C₆-alkylsulphinyl-, C₁-C₆-haloalkylsulphinyl-, C₁-C₆-alkylsulphonyl-, C₁-C₆-haloalkylsulphonyl-, C₂-C₆-alkenylthio-, C₂-C₆-haloalkenyl-thio-, C₂-C₆-alkynylthio-, C₃-C₆-cycloalkylthio-, C₃-C₆-cycloalkyl-C₁-C₆-alkylthio-, C₁-C₆-alkylamino-, C₁-C₆-alkylamino-carbonyl-, di(C₁-C₆-alkyl)amino-, di(C₁-C₆-alkyl)amino-carbonyl-, C₁-C₆-alkyl-carbonylamino-, C₁-C₆-haloalkyl-carbonylamino-, C₁-C₆-alkoxy-carbonylamino-, C₁-C₆-alkyl-aminocarbonylamino-substituted, or phenyl-, phenyloxy-, benzyl-, benzyloxy-, phenylamino- or benzylamino-substituted (where in each case the phenyl groups are optionally substituted by nitro, hydroxyl, mercapto, amino, cyano, C₁-C₆-alkyl, C₁-C₆-haloalkyl, C₁-C₆-alkyl-carbonyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-haloalkenyloxy, C₂-C₆-alkynyloxy, C₁-C₆-alkylthio, C₁-C₆-alkylsulphinyl, C₁-C₆-alkylsulphonyl or C₁-C₆-alkoxy-carbonyl) aryl or monocyclic or bicyclic heteroaryl having up to 10 carbon atoms and at least one heteroatom from the group N (nitrogen, 1 to 5 N atoms), O (oxygen, 1 or 2 O atoms), sulphur (1 or 2 S atoms) and optionally, as a replacement or in addition, an SO or SO₂ moiety, and optionally in addition a carbonyl moiety (C═O) and/or a thiocarbonyl moiety (C═S) as part of the heterocycle.
R⁶ is further one of the following moieties:

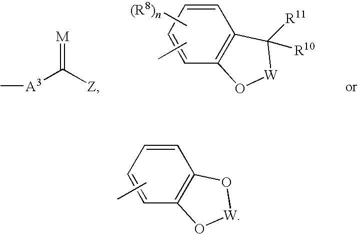

A³ is a bond or is a C₁-C₆ alkylene bridge which is optionally substituted one or more times by halogen or by C₃-C₈ cycloalkyl.
M is oxygen or NOR⁷.
R⁷ is hydrogen, C₁-C₁₂alkyl, C₃-C₈cycloalkyl, C₁-C₆alkylcarbonyl, C₂-C₆-alkenyl, C₂-C₆alkynyl, aryl, heterocyclyl or benzyl, with alkyl, cycloalkyl, alkenyl and alkynyl radicals being unsubstituted or being optionally substituted one to five times by a radical from the following group: halogen, —N₃, CN, NO₂, OH, SH, C₁-C₆alkoxy, C₁-C₆haloalkoxy, C₂-C₆alkenyloxy, C₂-C₆-haloalkenyloxy, C₃-C₆-alkynyloxy, C₃-C₆haloalkynyloxy, C₃-C₈cycloalkyl-C₁-C₆alkoxy, C₁-C₆-alkylcarbonyl, C₁-C₆haloalkylcarbonyl, C₁-C₆alkoxycarbonyl, C₁-C₆alkylcarbonyl-C₁-C₆alkyl, C₁-C₆alkoxycarbonyl-C₁-C₆alkyl, C₁-C₆alkylthio, C₂-C₆alkylthio, C₃-C₆alkynylthio, C₃-C₆cycloalkyl-C₁-C₆alkylthio, C₃-C₆haloalkynyl, C₂-C₆haloalkenylthio, C₁-C₆haloalkylthio, C₁-C₆alkoxy-C₁-C₆alkyl, C₁-C₆haloalkoxy-C₁-C₆alkyl, C₂-C₆alkenyloxy-C₁-C₆alkyl, C₂-C₆haloalkenyloxy-C₁-C₆alkyl, C₃-C₆alkynyloxy-C₁-C₆alkyl, NH₂, NHC₂-C₆alkenyl, C₂-C₆haloalkenyl, C₃-C₆alkynyl, C₃-C₈cycloalkyl, C₃-C₈cycloalkyl-C₁-C₆alkyl, C₁-C₆alkoxy, C₁-C₆haloalkoxy, C₂-C₆alkenyloxy, C₂-C₆haloalkenyloxy, C₃-C₆alkynyloxy, C₃-C₆haloalkynyloxy, C₃-C₈cycloalkyl-C₁-C₆alkoxy, C₁-C₆alkylcarbonyl, C₁-C₆haloalkyl-carbonyl, C₁-C₆alkoxycarbonyl, C₁-C₆alkylcarbonyl-C₁-C₆-alkyl, C₁-C₆alkoxycarbonyl-C₁-C₆alkyl, C₁-C₆alkylthio, C₂-C₆alkenylthio, C₃-C₆alkynylthio, C₃-C₆cycloalkyl-C₁-C₆alkylthio, C₃-C₆haloalkynyl, C₂-C₆haloalkenylthio, C₁-C₆haloalkylthio, C₁-C₆alkoxy-C₁-C₆alkyl, C₁-C₆haloalkoxy-C₁-C₆alkyl, C₂-C₆alkenyloxy-C₁-C₆alkyl, C₂-C₆haloalkenyloxy-C₁-C₆alkyl, C₃-C₆alkynyloxy-C₁-C₆alkyl, N(C₁-C₆alkyl)₂, the two alkyl groups independently of one another being able to be C₁-C₆alkylcarbonylamino, C₁-C₆haloalkylcarbonylamino, C₁-C₆alkoxycarbonylamino and C₁-C₆alkylaminocarbonylamino.
W is *C(R³²R³³) or *C(R³²R³³)C(R³⁴R³⁵), where the asterisk “*”
   denotes the attachment to oxygen, and in which R³² to R³⁵ each independently of one another are halogen, C₁-C₄alkyl, C₁-C₄alkoxy(C₁-C₄)alkyl or halo(C₁-C₄)alkyl.
n can adopt values from 0 to 3,
R⁸ is independently at each occurrence hydrogen, halogen, halo(C₁-C₄)alkyl, C₃-C₆cycloalkyl, C₂-C₅alkenyl, C₂-C₅alkynyl, C₁-C₄haloalkyl, C₁-C₄alkoxy, C₁-C₄haloalkoxy, C₁-C₄alkylthio, C₁-C₄haloalkylthio, C₁-C₄alkylsulphonyl, C₁-C₄haloalkylsulphonyl, nitro, cyano, aryl, alkylcarbonylamino, arylcarbonylamino and C₁-C₄alkoxycarbonylamino.
R¹⁰ and R¹¹ are in each case independently of one another hydrogen, halogen, hydroxyl, (C₁-C₄) alkyl, halo(C₁-C₄)alkyl or (C₁-C₄)alkoxy or together are carbonyl, are —O—CH₂—CH₂—O—, S—CH₂—CH₂—S, ketal, thioketal or forming an oxime in the form NOR², where
R¹² is (C₁-C₃)alkyl, (C₁-C₃)alkoxy(C₁-C₃)alkyl, aryl-(C₁-C₃)alkyl, C₂-C₄alkenyl-(C₁-C₃)alkyl, halo(C₂-C₄)alkenyl-(C₁-C₃)alkyl, di(C₁-C₃)alkylphosphonate, formyl, (C₁-C₃)alkylcarbonyl, halo(C₁-C₃)alkylcarbonyl, (C₁-C₃)alkoxy(C₁-C₃)alkylcarbonyl, arylcarbonyl and (C₁-C₃)alkylsulphonyl.

Preferred substituents and preferred ranges of the radicals present in the formulae recited above and below are defined in the following text.
A¹ is preferably one of the following moieties:
   —CH₂—CH═CCl₂, —CH₂—CH═CBr₂, —CH₂—CH═CClF, —CH₂—CH═CBrCl, —CH₂—CH═CBrF.
A¹ is with particular preference one of the following moieties:
   —CH₂—CH═CCl₂, —CH₂—CH═CBr₂, —CH₂—CH═CBrCl.
A¹ is with very particular preference the moiety —CH₂—CH═CCl₂.
A²-X is preferably a straight-chain or branched alkanediyl or alkenediyl having in each case up to 8 carbon atoms, which optionally within the carbon chain contains an oxygen atom, sulphur atom or a moiety selected from SO, SO₂, NH, N(C₁-C₄-alkyl), and also is
   —(C₁-C₄-alkyl)-(CO)—O—, —(C₁-C₄-alkyl)-(CO)—(NH)—, —(C₁-C₄-alkyl)-(CO)-[N—(C₁-C₄-alkyl)]-, —(C₁-C₄-alkyl)-(CO)—S—, (C₂-C₈-alkenyl)-(CO)—O—, —(C₂-C₈-alkenyl)-(CO)—(NH)—, —(C₂-C₈-alkenyl)-(CO)-[N—(C₁-C₄-alkyl)]-, —(C₂-C₈-alkenyl)-(CO)—S, piperidyl or piperazinyl, —(C₁-C₄-alkyl)-O—N═C(R⁹)—, —(C₁-C₆-alkenyl)-O—N═C(R⁹)—, in which
R⁹ preferably is methyl or is aryl.
A²-X is with particular preference a straight-chain or branched alkanediyl or alkenediyl having in each case up to 8 carbon atoms, which optionally within the carbon chain contains an oxygen atom, and also is
   —(C₁-C₄-alkyl)-(CO)—O—, —(C₁-C₄-alkyl)-(CO)—(NH)—, —(C₁-C₄-alkyl)-(CO)-[N—(C₁-C₄-alkyl)]-, —(C₁-C₄-alkyl)-(CO)—S—, (C₂-C₈-alkenyl)-(CO)—O—, —(C₂-C₈-alkenyl)-(CO)—(NH)—, —(C₂-C₈-alkenyl)-(CO)-[N—(C₁-C₄-alkyl)]-, —(C₂-C₈-alkenyl)-(CO)—S, piperidyl or piperazinyl, —(C₁-C₄-alkyl)-O—N═C(R⁹)—, —(C₁-C₆-alkenyl)-O—N═C(R⁹)—, in which
R⁹ is with particular preference methyl.
A²-X is with very particular preference a straight-chain or branched alkanediyl having up to 8 carbon atoms, which optionally within the carbon chain contains an oxygen atom, and also is —(C₁-C₄-alkyl)-(CO)—O—, —(C₁-C₄-alkyl)-(CO)—(NH)— or piperidyl.

Within the above definition of the optional bridge

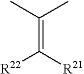

the following definitions of the radicals R²¹ and R²² are preferred or particularly preferred.
R²¹ and R²² are independently of one another preferably hydrogen, nitro, hydroxyl, amino, cyano, cyanato, thiocyanato, formyl, halogen or methyl,
R²¹ and R²² are with particular preference hydrogen.
R¹ is preferably hydrogen, nitro, hydroxyl, cyano, fluoro, chloro, bromo, methyl, ethyl, n- or isopropyl, methoxy, ethoxy, n- or isopropoxy, methylthio, ethylthio, n- or isopropylthio, methylamino, ethylamino, n- or isopropylamino, dimethylamino or is the moiety —O-A¹, where A¹ has one of the definitions indicated above.
R¹ is with particular preference hydrogen, nitro, hydroxyl, cyano, fluoro, chloro, bromo, methyl, ethyl, n- or isopropyl, methoxy, ethoxy, n- or isopropoxy, methylthio, ethylthio, n- or isopropylthio, methylamino, ethylamino, n- or isopropylamino or dimethylamino.
R¹ is with very particular preference hydrogen, methyl, ethyl, methoxy, ethoxy or fluoro, chloro, bromo.
R² is preferably hydrogen, nitro, cyano, cyanato, thiocyanato, formyl, halogen, is in each case optionally cyano-, halogen- or C₁-C₅-alkoxy-substituted alkyl, alkoxy, alkylthio, alkylamino, dialkylamino or alkylcarbonylamino having in each case 1 to 5 carbon atoms in the alkyl groups, is C₁-C₅-alkyl-carbonyl, C₁-C₅-alkoxy-carbonyl, C₁-C₅-alkoximinoformyl, C₁-C₅-alkoximino-acetyl, or is C₂-C₅-alkenyl or C₂-C₅-alkynyl.
R² is with particular preference hydrogen, nitro, cyano, cyanato, thiocyanato, formyl, fluoro, chloro, bromo, iodo, is in each case optionally cyano-, fluoro-, chloro-, methoxy-, ethoxy-, n- or isopropoxy-substituted methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino, dimethylamino, diethylamino, acetylamino, propionylamino, n- or isobutyroylamino, acetyl, propionyl, n- or isobutyroyl, methoxycarbonyl, ethoxycarbonyl, n- or isopropoxycarbonyl, methoximinoformyl, ethoximinoformyl, methoximinoacetyl or ethoximinoacetyl.
R² is with very particular preference hydrogen, cyano, fluoro, chloro, bromo or iodo.
R² is most preferably hydrogen, fluoro, chloro or bromo.
R³ is preferably hydrogen, nitro, halogen, is in each case optionally cyano-, halogen- or C₁-C₅-alkoxy-substituted alkyl, alkoxy, alkylthio or alkylamino having in each case 1 to 5 carbon atoms in the alkyl groups.

R³ is with particular preference hydrogen, nitro, fluoro, chloro, bromo, iodo, is in each case optionally cyano-, fluoro-, chloro-, methoxy-, ethoxy-, n- or isopropoxy-substituted methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino.
R³ is with very particular preference hydrogen, cyano, fluoro, chloro, bromo, methyl, ethyl, methoxy or ethoxy.
R⁴ is preferably hydrogen, nitro, halogen, is in each case optionally cyano-, halogen- or C₁-C₅-alkoxy-substituted alkyl, alkoxy, alkylthio or alkylamino having in each case 1 to 5 carbon atoms in the alkyl groups.
R⁴ is with particular preference hydrogen, nitro, fluoro, chloro, bromo, iodo, is in each case optionally cyano-, fluoro-, chloro-, methoxy-, ethoxy-, n- or isopropoxy-substituted methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino.
R⁴ is with very particular preference hydrogen, cyano, fluoro, chloro or bromo.
R⁵ is preferably an alkanyl moiety or alkynyl moiety having in each case 2 to 5 carbon atoms or cycloalkanyl moiety having 4 to 6 carbon atoms, which contain in each case at least one substituent from the group nitro, cyano, carboxyl, carbamoyl, hydroxyl, carbonyl (C═O), hydroximino (C═N—OH), C₁-C₅-alkoxy, C₁-C₅-alkoxy-carbonyl, C₁-C₅-alkylamino, di(C₁-C₄-alkyl)-amino, C₁-C₅-alkylamino-carbonyl, C₁-C₅-alkoxy-carbonylamino, C₁-C₅-alkoxy-C₁-C₅-alkoxy, C₁-C₅-alkoxyimino, C₃-C₅-alkenyloxy, C₃-C₅-alkynyloxy, C₃-C₅-alkenyloxy-carbonyl, C₃-C₅-alkynyloxy-carbonyl, C₃-C₅-alkenyloxyimino, C₃-C₅-alkynyloxyimino, C₃-C₆-cycloalkyl.
R⁵ is with particular preference hydrogen, methyl, isopropyl, cyclopropyl or cyclohexyl.
R⁵ is with very particular preference methyl.
R⁶ is preferably an alkenyl moiety or alkynyl moiety having in each case 2 to 6 carbon atoms or cycloalkenyl moiety having 4 to 6 carbon atoms, which is optionally substituted by at least one substituent from the group nitro, cyano, carboxyl, carbamoyl, hydroxyl, carbonyl (C═O), hydroximino (C═N—OH), C₁-C₄-alkoxy, C₁-C₄-alkoxy-carbonyl, C₁-C₄-alkylamino, di(C₁-C₄-alkyl)-amino, C₁-C₄-alkylamino-carbonyl, C₁-C₄-alkoxy-carbonylamino, C₁-C₄-alkoxy-C₁-C₆-alkoxy, C₁-C₄-alkoxyimino, C₃-C₄-alkenyloxy, C₃-C₄-alkynyloxy, C₃-C₄-alkenyloxy-carbonyl, C₃-C₄-alkynyloxy-carbonyl, C₃-C₄-alkenyloxyimino, C₃-C₅-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl.

Preference or particular preference is also given to the above-stated alternatives in the definition of R⁶ in which the radicals A³, Z, M, R⁷, W, n, R⁸, R¹⁰ and R¹¹ have the following preferred or particularly preferred definitions.
A³ is preferably a single bond or is straight-chain or branched and optionally halogen- or C₃-C₆-cycloalkyl-substituted alkanediyl having 1 to 6 carbon atoms,
A³ is with particular preference a single bond or is in each case optionally fluoro-, chloro-, bromo-, cyclopropyl-, cyclobutyl-, cyclopentyl- or cyclohexyl-substituted methylene, ethane-1,1-diyl (ethylidene), ethane-1,2-diyl (dimethylene), propane-1,1-diyl (propylidene), propane-1,2-diyl, propane-1,3-diyl (trimethylene), butane-1,1-diyl (butylidene) or butane-1,4-diyl (tetramethylene),
A³ is with very particular preference a single bond or is methylene,
Z is preferably in each case optionally nitro-, hydroxyl-, amino-, formyl-, cyano-, carboxyl-, carbamoyl-, fluoro-, chloro-, bromo-, methyl-, ethyl-, n- or isopropyl-, n-, iso-, s- or t-butyl-, fluoromethyl-, chloromethyl-, difluoromethyl-, dichloromethyl-, trifluoromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluoropropyl-, chloropropyl-, difluoropropyl-, dichloropropyl-, trifluoropropyl-, fluoroisopropyl-, difluoroisopropyl-, trifluoroisopropyl-, tetrafluoroisopropyl-, pentafluoroisopropyl-, methoxy-, ethoxy-, n- or isopropoxy-, fluoromethoxy-, difluoromethoxy-, trifluoromethoxy-, fluorodichloromethoxy-, chlorodifluoromethoxy-, fluoroethoxy-, chloroethoxy-, difluoroethoxy-, dichloroethoxy-, trifluoroethoxy-, fluoropropoxy, methoxycarbonyl-, ethoxycarbonyl-, fluoroethoxycarbonyl-, chloroethoxycarbonyl-, methoxymethyl-, ethoxymethyl-, n- or isopropoxymethyl-, methoxyethyl-, ethoxyethyl-, n- or isopropoxyethyl-, ethenyl-, propenyl-, butenyl-, fluoroethenyl-, chloroethenyl-, difluoroethenyl-, dichloroethenyl-, trifluoroethenyl-, trichloroethenyl-, propenyloxymethyl-, butenyloxymethyl-, propenyloxyethyl-, butenyloxyethyl-, ethynyl-, propynyl-, butynyl-, propenyloxy-, butenyloxy-, fluoropropenyloxy-, chloropropenyloxy-, fluorobutenyloxy-, chlorobutenyloxy-, propenyloxycarbonyl-, butenyloxycarbonyl-, propynyloxy-, butynyloxy-, propynyloxycarbonyl-, butynyloxycarbonyl-, propynyloxymethyl-, butynyloxymethyl-, propynyloxyethyl-, butynyloxyethyl-, cyclopropyl-, cyclobutyl-, cyclopentyl-, cyclopropylcarbonyl-, cyclobutylcarbonyl-, cyclopentylcarbonyl-, cyclopropylmethyl-, cyclobutylmethyl-, cyclopentylmethyl-, cyclohexylmethyl-, cyclopropylmethylcarbonyl-, cyclobutylmethylcarbonyl-, cyclopentylmethylcarbonyl-, cyclopropyloxy-, cyclobutyloxy-, cyclopentyloxy-, cyclohexyloxy-, cyclopropyloxycarbonyl-, cyclobutyloxycarbonyl-, cyclopentyloxycarbonyl-, cyclohexyloxycarbonyl-, cyclopropylmethoxy-, cyclobutylmethoxy-, cyclopentylmethoxy-, cyclohexylmethoxy-, cyclopropylmethoxycarbonyl-, cyclobutylmethoxycarbonyl-, cyclopentylmethoxycarbonyl-, cyclohexylmethoxycarbonyl-, cyclopropylmethoxymethyl-, cyclobutylmethoxymethyl-, cyclopentylmethoxymethyl-, cyclopropyloxymethoxy-, cyclobutyloxymethoxy-, cyclopentyloxymethoxy-, acetylmethyl-, propionylmethyl-, n- or isobutyroylmethyl-, acetylethyl-, propionylethyl-, methoxycarbonylmethyl-, ethoxycarbonylmethyl-, n- or isopropoxycarbonylmethyl-, methoxycarbonylethyl-, ethoxycarbonylethyl-, n- or isopropoxycarbonylethyl-, methylthio-, ethylthio-, n- or isopropylthio-, difluoromethylthio-, trifluoromethylthio-, chlorodifluoromethylthio-, methylsulphinyl-, ethylsulphinyl-, trifluoromethylsulphinyl-, methylsulphonyl-, ethyl-sulphonyl-, trifluoromethylsulphonyl-, propenylthio-, butenylthio-, fluoropropenylthio-, chloropropenylthio-, fluorobutenylthio-, chlorobutenylthio-, propynylthio-, butynylthio-, cyclopropylthio-, cyclobutylthio-, cyclopentylthio-, cyclohexylthio-, cyclopropylmethylthio-, cyclobutylmethylthio-, cyclopentylmethylthio-, methylamino-, ethylamino-, n- or isopropylamino-, methylaminocarbonyl-, ethylaminocarbonyl-, n- or isopropylaminocarbonyl-, dimethylamino-, diethylamino-, dimethylaminocarbonyl-, diethylaminocarbonyl-, acetylamino-, propionylamino-, n- or isobutyroylamino-, methoxycarbonylamino-, ethoxycarbonylamino-, n- or isopropoxycarbonylamino-, methylaminocarbonylamino-, ethylaminocarbonylamino-, n- or isopropylaminocarbonylamino-substituted, or phenyl-, phenyloxy-, benzyl-, benzyloxy-, phenylamino- or benzylamino- (where in each case the phenyl groups are optionally substituted by nitro, hydroxyl, mercapto, amino, cyano, methyl, ethyl, n- or isopropyl, n-, iso-, s- or t-butyl, trifluoromethyl, methoxy, ethoxy, n- or isopropoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulphinyl, ethylsulphinyl, methylsulphonyl, ethylsulphonyl, methoxycarbonyl, ethoxycarbonyl, n- or isopropoxycarbonyl) monocyclic heteroaryl having up to 5 carbon atoms and at least one heteroatom from the group N (nitrogen, 1 to 4 N atoms), O (oxygen, 1 O atom), sulphur (1 S atom) and optionally, in replacement or in addition, an SO or SO₂ moiety, and optionally in addition a carbonyl moiety (C═O) and/or a thiocarbonyl moiety (C═S) as part of the heterocycle, mention being made in particular, as heteroaryl moieties, of pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl, but especially tetrazolyl.
M is preferably oxygen,
R⁷ is preferably hydrogen; C₁-C₆alkyl, C₃-C₆cycloalkyl, C₁-C₄alkylcarbonyl, C₂-C₄alkenyl, C₂-C₄alkynyl, aryl, heterocyclyl or benzyl, where alkyl, cycloalkyl, alkenyl and alkynyl radicals are unsubstituted or are optionally substituted one to five times by a radical from the following group: halogen, —N₃, CN, NO₂, OH, SH, C₁-C₄alkoxy, C₁-C₄haloalkoxy, C₂-C₄alkenyloxy, C₂-C₄haloalkenyloxy, C₃-C₄alkynyloxy, C₃-C₄haloalkynyloxy, C₃-C₆cycloalkyl-C₁-C₄alkoxy, C₁-C₄alkylcarbonyl, C₁-C₄haloalkylcarbonyl, C₁-C₄alkoxycarbonyl, C₁-C₄alkylcarbonyl-C₁-C₄alkyl, C₁-C₄alkoxycarbonyl-C₁-C₄alkyl, C₁-C₄alkylthio, C₂-C₄alkylthio, C₃-C₄alkynylthio, C₃-C₄cycloalkyl-C₁-C₄alkylthio, C₃-C₄haloalkynyl, C₂-C₄haloalkenylthio, C₁-C₄haloalkylthio, C₁-C₄alkoxy-C₁-C₄Alkyl, C₁-C₄haloalkoxy-C₁-C₄alkyl, C₂-C₄alkenyloxy-C₁-C₄alkyl, C₂-C₄haloalkenyloxy-C₁-C₄alkyl, C₃-C₄alkynyloxy-C₁-C₄alkyl, NH₂, NHC₂-C₄alkenyl, C₂-C₄haloalkenyl, C₃-C₆alkynyl, C₃-C₆cycloalkyl, C₃-C₆cycloalkyl-C₁-C₄alkyl, C₁-C₄alkoxy, C₁-C₄haloalkoxy, C₂-C₄alkenyloxy, C₂-C₄haloalkenyloxy, C₃-C₄alkynyloxy, C₃-C₄haloalkynyloxy, C₃-C₆cycloalkyl-C₁-C₄alkoxy, C₁-C₄alkylcarbonyl, C₁-C₄haloalkyl-carbonyl, C₁-C₄alkoxycarbonyl, C₁-C₄alkylcarbonyl-C₁-C₄alkyl, C₁-C₄alkoxycarbonyl-C₁-C₄alkyl, C₁-C₄alkylthio, C₂-C₄alkenylthio, C₃-C₄alkynylthio, C₃-C₆cycloalkyl-C₁-C₄alkylthio, C₃-C₄haloalkynyl, C₂-C₄haloalkenylthio, C₁-C₄haloalkylthio, C₁-C₄alkoxy-C₁-C₆alkyl, C₁-C₄haloalkoxy-C₁-C₄alkyl, C₂-C₄alkenyloxy-C₁-C₄alkyl, C₂-C₄haloalkenyloxy-C₁-C₄alkyl, C₃-C₄alkynyloxy-C₁-C₄alkyl, N(C₁-C₄alkyl)₂, where the two alkyl groups each independently of one another may be C₁-C₄alkylcarbonylamino, C₁-C₄haloalkylcarbonylamino, C₁-C₄-alkoxycarbonylamino and C₁-C₄alkylaminocarbonylamino.
W is preferably *C(R³²R³³) and *C(R³²R³³)C(R³⁴R³⁵), where the asterisk “*”
   denotes the attachment to O and in which R³² to R³⁵ independently and in each case are halogen, C₁-C₃alkyl, C₁-C₃alkoxy(C₁-C₃)alkyl or halo(C₁-C₃)alkyl,
W is with particular preference C(R₃₂R₃₃), where R₃₂R₃₃ is halogen or C₁-C₄alkyl.
W is with very particular preference C(R₃₂R₃₃), where R₃₂R₃₃ is halogen or methyl.
n preferably adopts values from 0 to 2.
n is with particular preference 0.
R⁸ is preferably, independently at each occurrence hydrogen, halogen, halo(C₁-C₄)alkyl or nitro.
R⁹ is with particular preference hydrogen.
R¹⁰ and R¹¹ are preferably, taken together, ═O or hydrogen.

Depending on factors which include the nature of the substituents, the compounds of the formula (I) may optionally be in the form of stereoisomers, i.e. in the form of geometrical and/or in the form of optical isomers or isomer mixtures in different compositions. Not only the pure stereoisomers but also any desired mixtures of these isomers are provided by this invention, despite reference being generally made only to the compounds of the formula (I).

The invention also provides the saltlike derivatives formed from compounds of the formula (I) by reaction with basic or acidic compounds.

Preferred substituents and preferred ranges of the radicals present in the formulae recited above and below are defined in the text below.

The general definitions of radicals recited above, or the above-recited definitions of radicals that are recited in ranges of preference, apply not only to the end products of the formula (I) but also, correspondingly, to the starting products or intermediates required in each case for their preparation. These definitions of radicals can be combined arbitrarily with one another, hence including arbitrary combinations of the preferred ranges specified.

Preference is given in accordance with the invention to those compounds of the formula (I) in which there is a combination of the definitions recited above as being preferred.

Particular preference is given in accordance with the invention to those compounds of the formula (I) in which there is a combination of the definitions recited above as being particularly preferred.

Very particular preference is given in accordance with the invention to those compounds of the formula (I) in which there is a combination of the definitions recited above as being very particularly preferred.

Maximum preference is given in accordance with the invention to those compounds of the formula (I) in which there is a combination of the definitions recited above as being most preferred.

In the definitions of radicals recited above and below, hydrocarbon radicals, such as alkyl, both alone and in conjunction with heteroatoms as in alkoxy, are in each case, as far as possible, linear or branched.

The new substituted aryl oximes of the general formula (I) exhibit advantageous biological properties. They are notable in particular for strong arthropodicidal (insecticidal and acaricidal) and nematicidal activity and can be used in agriculture, in forestry, in the protection of stored and other materials, and in the hygiene sector.

The new substituted aryl oximes of the general formula (I) can be prepared in accordance with the following general reaction scheme. The synthesis of the starting compounds of the formula (II) is described in DE 103 20 782 A1.

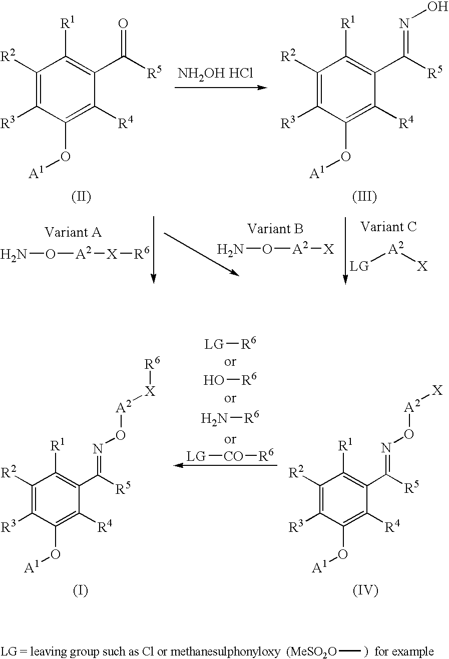

The precursors of the general formula H₂N—O-A²-X—R⁶ are obtainable via the following reaction pathway:

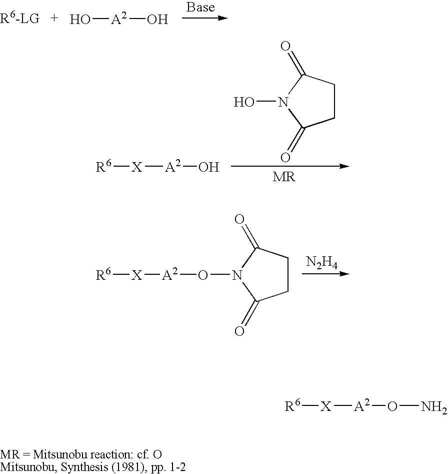

In the event that the bridge

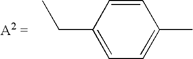

the general reaction scheme for the synthesis of compounds of the general formula (I) is as follows:

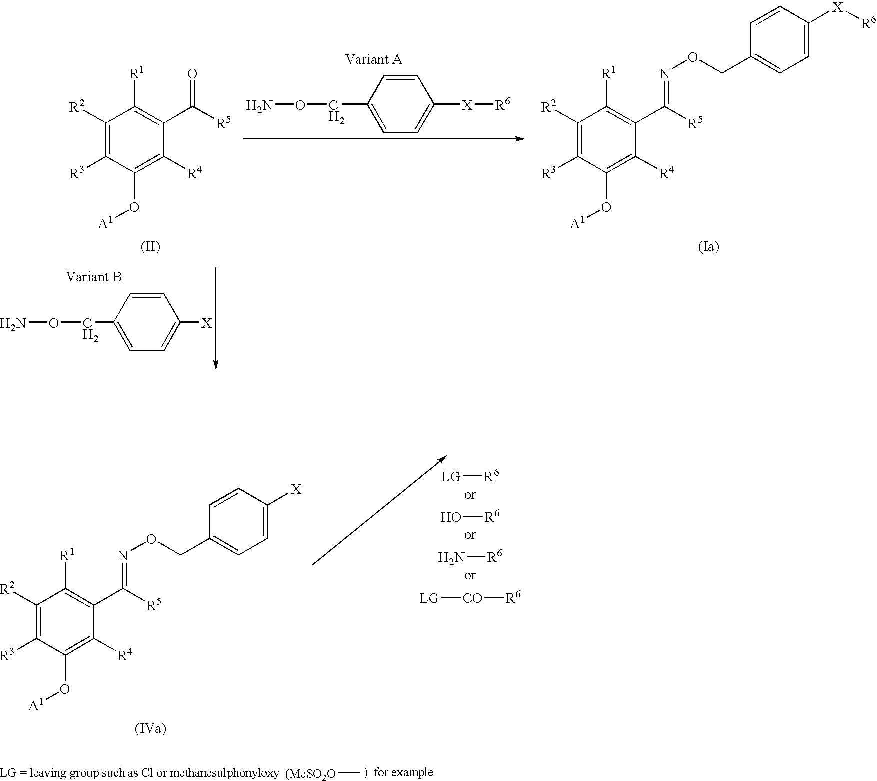

In the event that the bridge

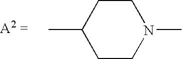

the general reaction scheme for the synthesis of compounds of the general formula (I) is as follows:

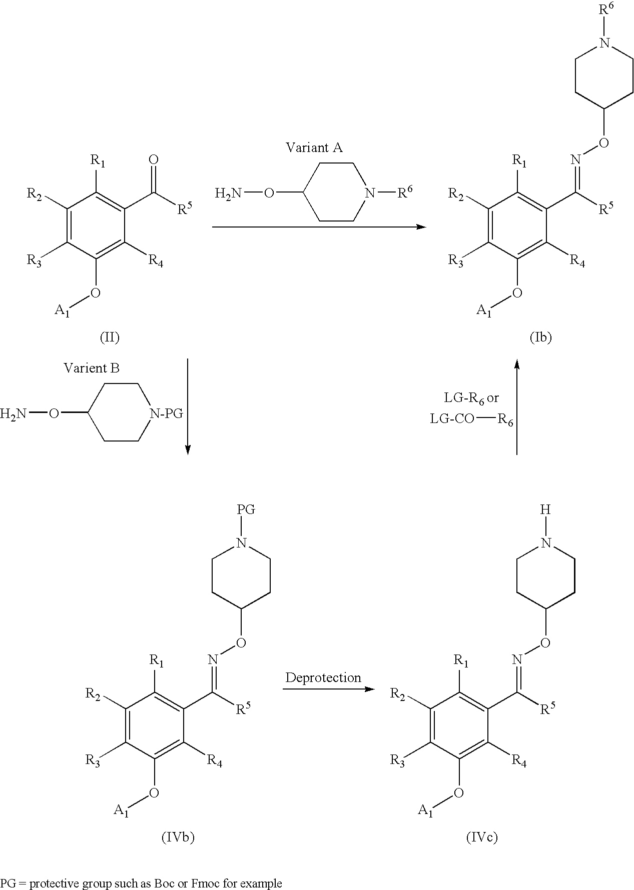

Optionally it is possible for the substituents of the compounds of the formula, such as the substituent R¹ for example, to be modified in further reaction steps as well. By way of example, in the event that R¹ is halogen, especially fluoro, a nucleophilic substitution with suitable nucleophils, within the bounds of the substituent definition of R¹, can be undertaken in the presence of basic reaction auxiliaries, which are specified below.

The starting materials of the general formula (II) are known and/or can be prepared by methods which are known per se (cf. Preparation Examples).

The reaction temperatures when the process of the invention is carried out can be varied within a relatively large range. Generally speaking, temperatures are employed of between 0° C. and 150° C., preferably between 10° C. and 120° C.

The process of the invention is generally carried out under atmospheric pressure. It is, however, also possible to carry out the process of the invention under elevated or reduced pressure—in general between 0.1 bar and 10 bar.

For the implementation of the process of the invention the starting materials are generally used in approximately equimolar amounts. It is, however, also possible to use one of the components in a relatively large excess. The reaction is generally carried out in a suitable diluent in the presence of a reaction auxiliary, and the reaction mixture is generally stirred at the required temperature for a number of hours. Working up is carried out in accordance with typical methods (cf. the Preparation Examples).

The compounds of the invention of the general formula (I) can be converted, by methods which are known in principle, into other compounds of the general formula (I). Some of these possible conversion reactions are outlined exemplarily below:

The compounds of the invention of the general formula (I) can form salts. Suitable salts of the compounds of the general formula (I) include typical non-toxic salts, i.e. salts with bases and salts (“adducts”) with acids. Preference is given to salts with inorganic bases, such as alkali metal salts, examples being sodium, potassium or caesium salts, alkaline earth metal salts, examples being calcium or magnesium salts, ammonium salts, salts with organic bases, especially with organic amines, examples being triethylammonium, dicyclohexylammonium, N,N′-dibenzylethylene-diammonium, pyridinium, picolinium or ethanolammonium salts, salts with inorganic acids, examples being hydrochlorides, hydrobromides, dihydrosulphates, trihydrosulphates, or phosphates, and salts with organic carboxylic acids or organic sulpho acids, examples being formates, acetates, trifluoroacetates, maleates, tartrates, methanesulphonates, benzenesulphonates or para-toluenesulphonates.

Salts are formed in accordance with the standard methods for salt preparation. For example the compounds of the invention are reacted with corresponding acids in order to form acid addition salts. Representative acid addition salts are salts which are formed, for example, through reaction with inorganic acids, such as sulphuric acid, hydrochloric acid, hydrobromic acid and phosphoric acid, for example, or with organic carboxylic acids such as acetic acid, trifluoroacetic acid, citric acid, succinic acid, lactic acid, formic acid, maleic acid, camphoric acid, phthalic acid, glycolic acid, glutaric acid, stearic acid, salicylic acid, sorbic acid, cinnamic acid, picric acid, benzoic acid or organic sulphonic acids such as methanesulphonic acid and para-toluenesulphonic acid.

The compounds of the formula (I) can, if appropriate, be present in different polymorphic forms or as a mixture of different polymorphic forms. Both the pure polymorphs and the polymorph mixtures are provided by the invention and can be used according to the invention.

The active compounds of the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as plant protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnostema consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogo-derma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuellebomi, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control Protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofinannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

The compounds of the formula (I) according to the invention have in particular excellent activity against . . . .

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, habitat or storage space by the customary treatment methods, for example by immersion, spraying, evaporation, fogging, scattering, painting on, injection and, in the case of propagation material, in particular in the case of seeds, also by applying one or more coats.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and microencapsulations in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared either in suitable plants or else before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulphoxide, and also water.

Suitable solid carriers are:

for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE- and/or -POP-ethers, acid and/or POP-POE esters, alkyl aryl and/or POP-POE ethers, fat- and/or POP-POE adducts, POE- and/or POP-polyol derivatives, POE- and/or POP-sorbitan- or -sugar adducts, alkyl or aryl sulphates, alkyl- or arylsulphonates and alkyl or arylphosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances, herbicides, safeners, fertilisers or semiochemicals.

Particularly favourable mixing components are, for example, the following compounds:

### Fungicides:

2-phenylphenol; 8-hydroxyquinoline sulphate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; quinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymi-done; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph, trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulphonyl)amino]butanamide; 1-(1-naphthalenyl)-1H-pyrrole-2,5-dione; 2,3,5,6-tetrachloro-4-(methylsulphonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedi-carbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro-[4.5]decan-3-amine; sodium tetrathiocarbonate; and also copper salts and preparations, such as Bordeaux mixture; copper hydroxide; copper naphthenate; copper oxychloride; copper sulphate; cufraneb; copper oxide; mancopper; oxine-copper.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### Insecticides/Acaricides/Nematicides:

Acetylcholine esterase (AChE) inhibitors
1.1 Carbamates,
   for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate
1.2 Organophosphates,
   for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

### Sodium Channel Modulators/Voltage-Dependent Sodium Channel Blockers

2.1 Pyrethroids,
   for example acrinathrin, allethrin (d-cis-trans, d-trans), beta-cyfluthrin, bifenthrin, bioallethrin, bioallethrin-S cyclopentyl isomer, bioethanomethrin, biopermethrin, bioresmethrin, chlovaportn, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, taufluvalinate, tefluthrin, terallethrin, tetramethrin (1R isomer), tralomethrin, transfluthrin, ZXI 8901, pyrethrins (pyrethrum)

DDT
2.2 Oxadiazines,
   for example indoxacarb

### Acetylcholine Receptor Agonists/Antagonists

3.1 Chloronicotinyls,
   for example acetamiprid, clothianidin, dinotefuran, imidacloprid, nitenpyram, nithiazine, thiacloprid, thiamethoxam
3.2 Nicotines, Bensultap, Cartap

### Acetylcholine Receptor Modulators

4.1 Spinosyns,
   for example spinosad

### GABA-Controlled Chloride Channel Antagonists

5.1 Organochlorines,
   for example camphechlor, chlordane, endosulfan, gamma-HCH, HCH, heptachlor, lindane, methoxychlor
5.2 Fiproles,
   for example acetoprole, ethiprole, fipronil, pyrafluprole, pyriprole, vaniliprole

### Chloride Channel Activators

6.1 Mectins,
   for example avermectin, emamectin, emamectin-benzoate, ivermectin, milbemycin

### Juvenile Hormone Mimetics,

for example diofenolan, epofenonane, fenoxycarb, hydroprene, kinoprene, methoprene, pyriproxifen, triprene

### Ecdysone Agonists/Disruptors

8.1 Diacylhydrazines,
   for example chromafenozide, halofenozide, methoxyfenozide, tebufenozide

### Chitin Biosynthesis Inhibitors

9.1 Benzoylureas,
   for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron
9.2 Buprofezin
9.3 Cyromazine

### Oxidative Phosphorylation Inhibitors, ATP Disruptors

10.1 Diafenthiuron
10.2 Organotin Compounds,
   for example azocyclotin, cyhexatin, fenbutatin-oxide

### Oxidative Phosphorylation Decouplers Acting by Interrupting the H-Proton Gradient

11.1 Pyrroles,
   for example chlorfenapyr
11.2 Dinitrophenols,
   for example binapacyrl, dinobuton, dinocap, DNOC

### Site-I Electron Transport Inhibitors

12.1 METIs,
   for example fenazaquin, fenpyroximate, pyrimidifen, pyridaben, tebufenpyrad, tolfenpyrad
12.2 Hydramethylnon
12.3 Dicofol

### Site-II Electron Transport Inhibitors

Rotenone
   Site-III electron transport inhibitors
Acequinocyl, fluacrypyrim

### Microbial Disruptors of the Insect Gut Membrane

Bacillus thuringiensis strains

### Fat Synthesis Inhibitors

Tetronic Acids,
for example spirodiclofen, spiromesifen
Tetramic Acids,
for example spirotetramat (CAS Reg. No.: 203313-25-1) and 3-(2,5-dimethylphenyl)-8-methoxy-2-oxo-1-azaspiro[4.5]dec-3-en-4-yl ethyl carbonate (aka: carbonic acid, 3-(2,5-dimethylphenyl)-8-methoxy-2-oxo-1-azaspiro[4.5]dec-3-en-4-yl ethyl ester, CAS Reg. No.: 382608-10-8)
Carboxamides,
for example flonicamid
Octopaminergic Agonists,
for example amitraz

### Inhibitors of Magnesium-Stimulated ATPase,

Propargite
activators of the ryanodine-sensitive calcium channel, for example
Benzoic Acid Dicarboxamides,
for example flubendiamides
Nereistoxin Analogues
for example thiocyclam hydrogen oxalate, thiosultap-sodium

### Biologicals, Hormones or Pheromones

azadirachtin, Bacillus spec., Beauveria spec., codlemone, Metarrhizium spec., Paecilomyces spec., thuringiensin, Verticillium spec.
   Active Compounds with Unknown or Unspecific Mechanisms of Action
23.1 Fumigants,
   for example aluminium phosphide, methyl bromide, sulfuryl fluoride
23.2. Antifeedants,
   for example cryolite, flonicamid, pymetrozine
23.3 Mite Growth Inhibitors,
   for example clofentezine, etoxazole, hexythiazox
23.4 Amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin

A mixture with other known active compounds, such as herbicides, fertilizers, growth regulators, safeners, semiochemicals, or else with agents for improving the plant properties, is also possible.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

The compounds are employed in a customary manner appropriate for the use forms.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The transgenic plants or plant cultivars (obtained by genetic engineering) which are preferably to be treated according to the invention include all plants which, by virtue of the genetic modification, received genetic material which imparted particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defence of the plants against insects, arachnids, nematodes and worms by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula I and/or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the Heteropterida, for example, Cimex spp., Triatoma spp., Rhodnius spp., Panstrongylus spp.

From the order of the Blattarida, for example, Blatta orientalis, Periplaneta americana, Blattela germanica, Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dennacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Emobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;

Hymenopterons, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, such as hulls, screens, nets, buildings, moorings and signalling systems, against fouling.

Furthermore, the compounds according to the invention, alone or in combination with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Pemphigus spp., Phylloera vastatrix, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric esters, carbamates, pyrethroids, neonicotinoides, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

### PREPARATION EXAMPLES

### Examples of Preparation of Precursors for the Synthesis of the Compounds of the General Formula (II)

### (1): 1-(3-Chloro-2,5-dihydroxyphenyl)ethanone

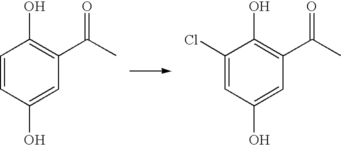

Over the course of 2 h 57 g of N-chlorosuccinimide are added in portions and under nitrogen to a solution of 50 g of 2,5-dihydroxypentylacetone in 800 ml of DMF.

The reaction solution undergoes brown discoloration and is stirred at RT overnight.

The reaction mixture is poured into 1.5 l of water and is extracted a number of times with a 1:1 hexane/ethyl acetate mixture. The combined organic phases are washed twice with water, dried over Na₂SO₄ and filtered and the filtrate is evaporated to dryness. The crude product is recrystallized from hexane/ethyl acetate. This gives 20.5 g of clean 1-(3-chloro-2,5-dihydroxyphenyl)ethanone and 28.5 g of mildly impure product.

M+(ES+)=(187, 100)

¹H-NMR (CD₃CN): 2.60 (s, 3H, CH₃); 6.98 (bs, 1H, OH); 7.18 (d, J=0.3 Hz, 1H, aryl); 7.25 (d, J=0.3 Hz, 1H, aryl); 12.22 (s, 1H, OH)

### (2): 1-(3-Chloro-2-hydroxy-5-triisopropylsilanyloxyphenyl)ethanone

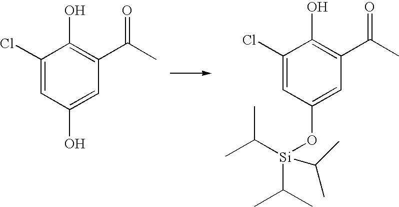

First 5 g of 1-(3-chloro-2,5-dihydroxyphenyl)ethanone in 300 ml of dichloromethane are admixed with 4 g of triethylamine and 5.2 g of triisopropylsilyl chloride and the reaction mixture is stirred at RT for 3 h. It is washed with water, dried over Na₂SO₄ and filtered and the filtrate is evaporated to dryness. This gives 8.28 g (in 73% pure form according to LC-MS) of 1-(3-chloro-2-hydroxy-5-triisopropylsilanyloxyphenyl)ethanone as a dark oil.

M+(ES+)=(343, 100)

¹H-NMR (CD₃CN): 1.11 (d, J=7.2 Hz; 9H, CH₃, iPr); 1.26-1.33 (m, 3H, CH, iPr); 2.60 (s, 3H, CH₃); 7.23 (d, J=2.9 Hz, 1H, aryl); 7.29 (d, J=2.9 Hz, 1H, aryl); 12.33 (s, 1H, OH).

In the same way as for (2) the following compounds were prepared:

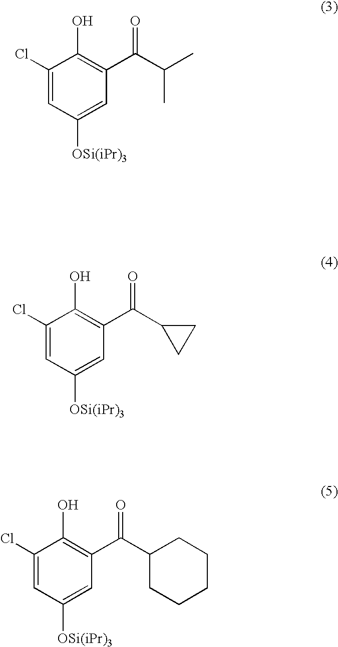

### (3): (3-Chloro-2-hydroxy-5-(triisopropylsilyloxy)phenyl)-2-methylpropan-1-one

Reaction of 33 g of 1-(3-chloro-2,5-dihydroxyphenyl)-2-methylpropan-1-one and 31.3 g of chlorotriisopropylsilane gave 56 g of product (93% pure according to LC-MS) of a brown oil.

M+(ES+)=(371, 100)

¹H-NMR (DMSO-D₆): 1.08 (d, J=7.3 Hz, 18H, Si-iPr₃) 1.14 (d, J=6.8 Hz, 6H, iPr); 1.25 (m, 3H, Si-iPr₃); 3.63 (m, 1H, iPr); 7.23 (d, J=2.9 Hz, 1H, aryl); 7.27 (d, J=2.9 Hz, aryl); 12.0 (s, 1H, OH)

### (4): 3-Chloro-2-hydroxy-5-(triisopropylsilyloxy)phenyl cyclopropyl ketone

Reaction of 85 g of 3-chloro-2,5-dihydroxyphenyl cyclopropyl ketone and 81 g of chlorotriisopropylsilane followed by brief column filtration (5:1 cyclohexane/ethyl acetate) gave 85 g of a brown oil product.

M+(ES+)=(369, 100)

¹H-NMR (DMSO-D₆): 1.08 (d, J=7.3 Hz, 18H, -iPr); 1.09 (m, 4H, -cPr); 1.26 (m, 3H, -iPr); 2.17 (m, 1H, -cPr); 7.24 (d, J=2.9 Hz, 1H, aryl); 7.27 (d, J=2.9 Hz, aryl); 11.85 (s, 1H, OH)

### (5): 3-Chloro-2-hydroxy-5-(triisopropylsilyloxy)phenyl cyclohexyl ketone

Reaction of 33 g of (3-chloro-2,5-dihydroxyphenyl)cyclohexyl ketone and 31.3 g of chlorotriisopropylsilane gave 56 g of a brown oil crude product, which was used without purification in the next step.

M+(ES+)=(411, 100)

¹H-NMR (DMSO-D₆): 1.08 (d, J=7.3 Hz, 18H, -iPr); 1.24 (m, 3H, -iPr); 1.33 (m, 2H, -cHex); 2.58-2.83 (bm, 8H, chex); 2.72 (m, 1H, -cHex); 7.25 (m, 2H, aryl); 12.0 (s, 1H, OH)

### (6): 1-(3-Chloro-5-hydroxy-2-methoxyphenyl)ethanone

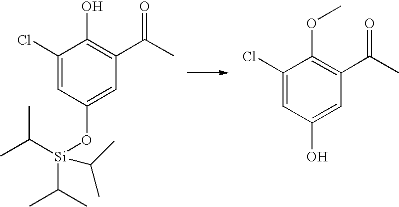

First 15 g of 1-(3-chloro-2-hydroxy-5-triisopropylsilanyloxyphenyl)ethanone are stirred into 250 ml of DMF together with 24 g of potassium carbonate and 24.8 g of dimethyl sulphate at RT for approximately 5 h. The reaction mixture is filtered and the residue is washed with dichloromethane. The combined organic phases are poured into water, stirred for 30 minutes thereafter and extracted three times with dichloromethane, the extracts being dried over Na₂SO₄ and filtered and the filtrate being evaporated to dryness.

The crude product is taken off in 250 ml of ethanol, 1N NaOH is added, and the mixture is stirred under reflux for a number of hours until a TLC check indicates complete conversion. The batch is concentrated to dryness, the residue is admixed with water, brought to a pH<7 with HCl and extracted a number of times with EE, the extracts are dried over Na₂SO₄ and filtered and the filtrate is evaporated to dryness on a rotary evaporator. The crude product is purified by flash chromatography over silica gel (gradient: 4:1 hexane/ethyl acetate to 100% ethyl acetate). This gives two product fractions: 4.6 g (100% purity according to LC-MS) and 1.3 g (95% purity according to LC-MS).

M+(ES+)=(201, 100)

¹H-NMR: 2.67 (s, 3H, CH₃); 3.85 (s, 3H, OCH₃); 6.21 (s, 1H, OH); 7.11 (d, J=6.2 Hz, 1H, aryl); 7.12 (d, J=6.2 Hz, 1H, aryl)

In the same way as for (6) the following compounds were prepared:

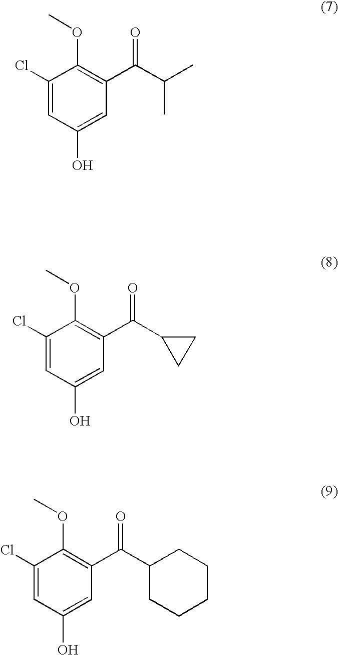

### (7): (3-Chloro-5-hydroxy-2-methoxyphenyl)-2-methylpropan-1-one

Reaction of 56.2 g of (3-chloro-2-hydroxy-5-(triisopropylsilyloxy)phenyl)-2-methylpropan-1-one and 28.6 ml of dimethyl sulphate gave a brown oil, which was stirred under reflux with 150 ml of 2N sodium hydroxide solution and 300 ml of ethanol for a number of hours and, following aqueous workup, was chromatographed over silica gel (5:1 cyclohexane/ethyl acetate). This gave 21.6 g of an orange-coloured oil (GC purity 71%).

M+(ES+)=(229, 100)

¹H-NMR (DMSO-D₆): 1.07 (d, J=6.9 Hz, 6H, -iPr); 3.26 (m, 1H, -iPr); 3.68 (s, 3H, —OMe); 6.75 (d, J=2.9 Hz, 1H, aryl); 6.98 (d, J=2.9 Hz, aryl); 9.88 (s, 1H, OH)

### (8): 3-Chloro-5-hydroxy-2-methoxyphenyl cyclopropyl ketone

Reaction of 85 g of 3-chloro-2-hydroxy-5-(triisopropylsilyloxy)phenyl cyclopropyl ketone and 58 g of dimethyl sulphate gave a brown oil, which was refluxed with 290 ml of 2N NaOH and 500 ml of ethanol for a number of hours and, following aqueous workup, was chromatographed on silica gel (5:1 cyclohexane/ethyl acetate). This gave 15.3 g of an orange-coloured oil.

M+(ES+)=(227, 100)

¹H-NMR (DMSO-D₆): 1.07 (d, J=6.2 Hz, 4H, -cPr); 2.61 (m, 1H, -cPr); 3.72 (s, 3H, —OMe); 6.84 (d, J=3 Hz, aryl); 7.02 (d, J=3 Hz, aryl); 9.92 (s, 1H, OH)

### (9): 3-Chloro-5-hydroxy-2-methoxyphenyl cyclohexyl ketone

Reaction of 83 g of 3-chloro-2-hydroxy-5-(triisopropylsilyloxy)phenyl cyclohexyl ketone and 51 g of dimethyl sulphate gave a brown oil, which was refluxed with 200 ml of 2N NaOH and 400 ml of ethanol for a number of hours and, following aqueous workup, was chromatographed on silica gel (5:1 cyclohexane/ethyl acetate). This gave 12 g of an orange-red oil.

M+(ES+)=(269, 100)

¹H-NMR (DMSO-D₆): 1.11-1.33 (bm, 4H, -cHex); 1.54-1.82 (bm, 6H, -cHex); 2.99 (m, 1H, -cHex); 3.68 (s, 3H, —OMe); 6.72 (d, J=2.9 Hz, aryl); 6.96 (d, J=2.9 Hz, aryl); 9.92 (s, 1H, OH)

### Examples of Preparation of Compounds of the General Formula (II)

### (10): 1-[3-Chloro-5-(3,3-dichloroallyloxy)-2-hydroxyphenyl]ethanone

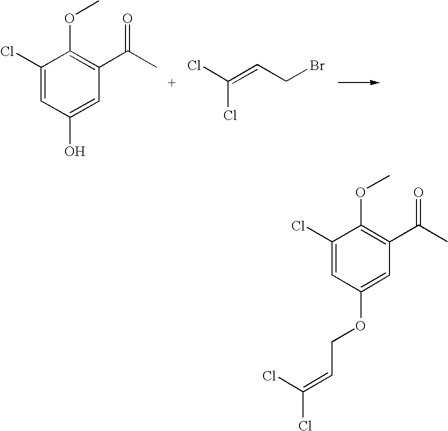

First 8.6 g of potassium carbonate in 200 ml of absolute dimethylformamide are admixed under nitrogen with 5 g of 1-(3-chloro-2,5-dihydroxyphenyl)ethanone. Subsequently 4.7 g of dichloropropenyl bromide are added dropwise with vigorous stirring, as a solution in 10 ml of DMF, and the batch is stirred at RT for a further 5 h. It is then poured into approximately 200 ml of water, extracted a number of times with ethyl acetate; the organic phase is washed with water, dried over Na₂SO₄ and filtered and the filtrate is evaporated to dryness. This gives 7.5 g (97% purity according to LC-MS) of 1-[3-chloro-5-(3,3-dichloroallyloxy)-2-hydroxyphenyl]ethanone as a viscose oil.

M+(ES+)=(309, 100)

¹H-NMR (CDCl₃): 2.65 (s, 3H, CH₃); 3.85 (s, 3H, OCH₃); 4.62 (d, J=6.3 Hz, 2H, CH₂); 6.12 (t, 1H, J=6.3 Hz, 1H, CH); 7.05 (d, J=3.2 Hz, 1H, CH aryl); 7.1 (d, J=3.2 Hz, 1H, CH aryl)

In analogy to the instructions for 1-[3-chloro-5-(3,3-dichloroallyloxy)-2-hydroxyphenyl]-ethanone (10) the following compounds were prepared:

| Identification | Structure | Physical data |
|---|---|---|
| (11): [3-Chloro-5-(3,3-dichloro- allyloxy)-2-methoxyphenyl]- cyclohexylmethanone | 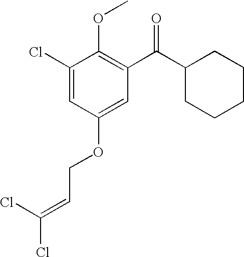 | M + (ES+) = (379, 100) ¹H-NMR(CD₃CN): 1.30-1.39 (m, 4H, cHex); 1.64-1.92 (m, 6H, cHex); 3.03-3.06 (m, 1H, cHex); 3.76 (s, 3H, OCH₃); 4.66 (d, J = 6.4 Hz, 2H, CH₂); 6.23 (t, J = 6.4 Hz, 1H, CH); 6.85 (d, J = 3.0 Hz, 1H, aryl); 7.12 (d, J = 3.0 Hz, 1H, aryl) |
| (12): 1-[3-Chloro-5-(3,3-dichloro- allyloxy)-2-methoxyphenyl]-2- methylpropan-1-one | 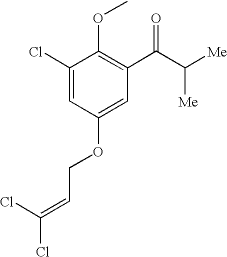 | M + (ES+) = (337, 100) ¹H-NMR(CDCl₃): 1.16 (d, J = 6.9 Hz, 6H, CH₃); 3.33-3.38 (m, 1H, iPr); 3.80 (s, 3H, OCH₃); 4.62 (d, J = 6.3 Hz, 2H, CH₂); 6.13 (t, J = 6.3 Hz, 1H, CH); 6.81 (d, J = 3.0 Hz, 1H, aryl); 7.03 (d, J = 3.0 Hz, 1H, aryl) |
| (13): [3-Chloro-5-(3,3-dichloro- allyloxy)-2-methoxyphenyl]- cyclopropylmethanone | 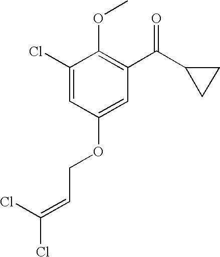 | M + (ES+): (335, 100) ¹H-NMR(CD₃CN): 1.06-1.11 (m, 2H, CH₂, cPr); 1.12-1.17 (m, 2H, CH₂, cPr); 2.62-2.68 (m, 1H CH, cPr); 4.68 (d, J = 6.4 Hz, 2H, CH₂); 6.26 (t, J = 6.4 Hz, 1H, CH); 7.00 (d, J = 3.1 Hz, 1H, aryl), 7.18 (d, J = 3.1 Hz, 1H, aryl) |

### Examples of Preparation of Compounds of the General Formula (III)

### (14): 1-[3-Chloro-5-(3,3-dichloroallyloxy)-2-methoxyphenyl]-2-methylpropan-1-one oxime

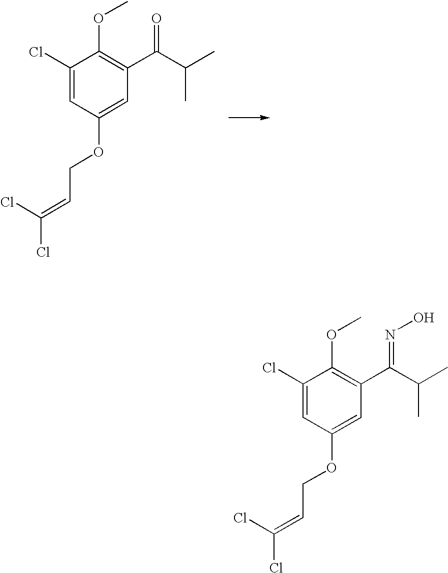

First 1 g of 1-[3-chloro-5-(3,3-dichloroallyloxy)-2-methoxyphenyl]-2-methylpropan-1-one are stirred together with 1.44 g of hydroxylammonium chloride and 1.7 g of sodium acetate in 10 ml of ethanol under reflux for approximately 3 hours. The reaction mixture is evaporated to dryness, the residue is extracted a number of times with ethyl acetate, the combined organic phases are dried over sodium sulphate and filtered and the filtrate is evaporated to dryness. This gives 1.06 g of 1-[3-chloro-5-(3,3-dichloroallyloxy)-2-methoxyphenyl]-2-methylpropan-1-one oxime as an E/Z isomer mixture.

M+(ES+)=(352, 100)

¹H-NMR (CD₃CN): 1.03 (m, 12 Hz, 4×CH₃, iPr); 2.67-2.74 (m, 1H, CH, iPr); 3.28-3.41 (m, 1H, CH, iPr); 3.71 (s, 3H, OCH₃); 3.72 (s, 3H, OCH₃); 4.64 (d, J=6.4 Hz, 2H, CH₂CHCCl₂); 4.65 (d, J=6.4 Hz, 2H, CH₂CHCCl₂); 6.25 (t, J=6.4 Hz, 2H, 2×CH₂CHCCl₂); 6.54 (d, J=3.0 Hz, aryl); 6.63 (d, J=3.0 Hz, aryl); 7.00 (d, J=3.0 Hz, aryl); 7.03 (d, J=3.0 Hz, aryl).

In analogy to the instructions for 1-[3-chloro-5-(3,3-dichloroallyloxy)-2-methoxyphenyl]-2-methylpropan-1-one oxime (14) the following compounds were prepared:

| Identification | Structure | Physical data |
|---|---|---|
| (15): [3-Chloro-5-(3,3- dichloroallyloxy)-2- methoxyphenyl]cyclo- hexylmethanone oxime | 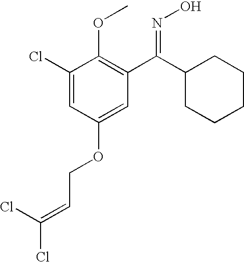 | M + (ES+) = (392, 100) ¹H-NMR(CD₃CN, E/Z isomers): 1.22-1.43 (m, H, cHex); 1.64-1.82 (m, H, cHex); 3.73 (s, 3H, OCH₃); 3.73 (s, 3H, OCH₃); 4.62-4.65 (m, 4H, 2x CH₂CHCCl₂); 6.15 (t, 1H, CH₂CHCCl₂); 6.22 (t, J = 6.4 1H, CH₂CHCCl₂); 6.51 (d, J = 3 Hz, 1H, aryl); 6.60 (d, J = 3 Hz, 1H, aryl); 6.98 (d, J = 3 Hz, 1H, aryl); 7.00 (d, J = 3 Hz, 1H, aryl); |
| (16): [3-Chloro-5-(3,3- dichloroallyloxy)-2- methoxyphenyl]- cyclopropylmethanone oxime | 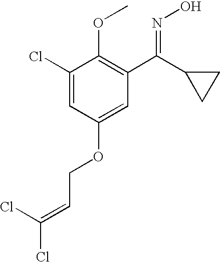 | M + (ES+) = (350, 90) ¹H-NMR(CD₃CN), E/Z isomers): 0.38-0.40 (m, 2H, CH₂, iPr); 0.59-0.61 (m, 2H, CH₂, iPr); 0.72-0.75 (m, 2H, CH₂, iPr); 0.80-0.83 (m, 2H, CH₂, iPr); 1.72-1.93, (m, 1H, CH); 2.43-2.46 (m, 1H, CH, iPr); 3.74 (s, 6H, 2x OCH₃); 4.63 (d, J = 6.4 Hz, 4H, 2x CH₂CHCCl₂); 6.24 (t, J = 6.4 Hz, 1H, CH₂CHCCl₂); 6.25 (t, J = 6.4 Hz, 1H, CH₂CHCCl₂); 6.53 (d, J = 3.0 Hz, aryl); 6.57 (d, J = 3.0 Hz, aryl); 7.00 (d, J = 3.0 Hz, aryl); 7.02 (d, J = 3.0 Hz, aryl). |

### (17): 1-[3-Chloro-5-(3,3-dichloroallyloxy)-2-methoxyphenyl]ethanone oxime

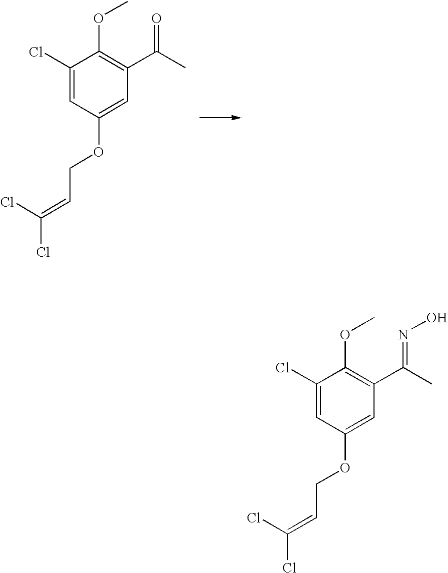

First 1 g of 2-methoxy-3-chloro-5-dichloropropenoxybenzylacetone in 4.3 ml of ethanol is admixed with 2.2 g of aqueous hydroxylamine solution (50% strength) and heated at 70° C. for 2 hours. The mixture is cooled to RT, the suspension formed is filtered, the solid is dissolved in dichloromethane, the solution is dried over sodium sulphate and filtered and the filtrate is evaporated to dryness. This gives 824 mg of 1-[3-chloro-5-(3,3-dichloroallyloxy)-2-methoxyphenyl]ethanone oxime as E/Z isomers in a ratio of 20:1.

M+(ES+)=(324, 100)

¹H-NMR (CD₃CN): 3.70 (s, 3H, OCH₃); 4.64 (d, J=6.4 Hz, 2H, CH₂CHCCl₂); 6.26 (t, J=6.4 Hz, 1H, CH₂CHCCl₂); 6.79 (d, J=3.0 Hz, 1H, aryl); 7.03 (d, J=3.0 Hz, 1H, aryl); 9.05 (s, 1H, OH).

The filtrate is evaporated to dryness, the residue is extracted twice with approximately 50 ml of dichloromethane, the extracts are dried over sodium sulphate and filtered and the filtrate is evaporated to dryness. This gives 209 mg of 1-[3-chloro-5-(3,3-dichloroallyloxy)-2-methoxyphenyl]ethanone oxime as E/Z isomers in a ratio of 1:1.

M+(ES+)=(324, 100)

### (18): 1-[3-Chloro-5-(3,3-dichloroallyloxy)-2-methoxyphenyl]ethanone O-(2-hydroxyethyl)-oxime

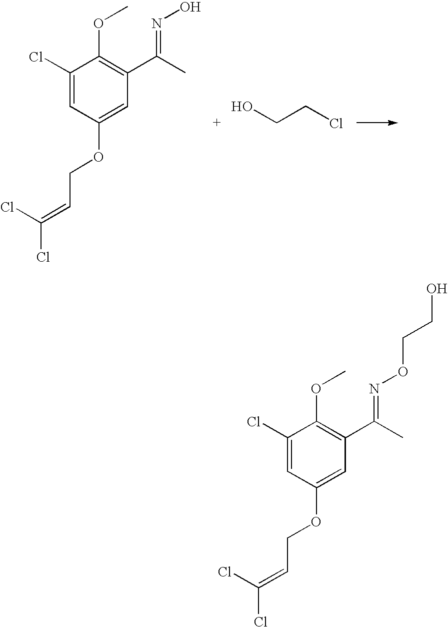

First 400 mg of 1-[3-chloro-5-(3,3-dichloroallyloxy)-2-hydroxyphenyl]ethanone are stirred together with 510 mg of potassium carbonate and 700 mg of 2-chloroethanol at 100° C. under nitrogen for 90 minutes. A further 345 mg of 2-chloroethanol are added and the mixture is stirred at 100° C. for a further 4 h.

The reaction mixture is admixed with approximately 10 ml of water and extracted 2× with approximately 50 ml of dichloromethane, the combined organic phases are dried over Na₂SO₄ and filtered and the filtrate is evaporated to dryness. This gives 560 mg of 1-[3-chloro-5-(3,3-dichloro-allyloxy)-2-methoxyphenyl]ethanone O-(2-hydroxyethyl)oxime.

M+(ES+)=(368, 25)

In the same way as for (18) the following compound is synthesized:

| Identification | Structure | Physical data |
|---|---|---|
| (19): (1E)-1-{3-Chloro-5-[(3,3- dichloroprop-2-en-1-yl)oxy]-2- methoxyphenyl}ethanone O-(4-hydroxybutyl)oxime | 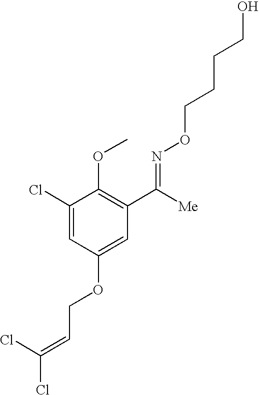 | (M + 396, 18) |

### Examples of Preparation of Compounds of the General Formula (I)

### (20): 1-[3-Chloro-5-(3,3-dichloroallyloxy)-2-methoxyphenyl]ethanone O-[2-(3-chloro-5-tri-fluoromethylpyridin-2-yloxy)ethyl]oxime

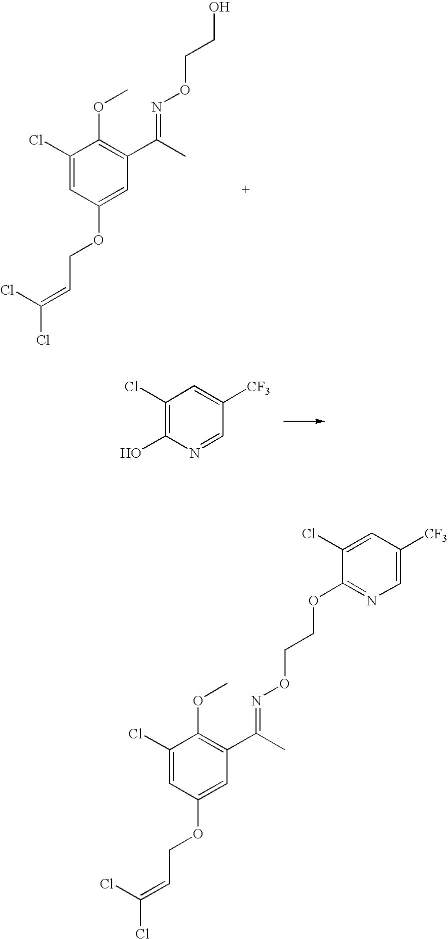

First 250 mg of 1-[3-chloro-5-(3,3-dichloroallyloxy)-2-methoxyphenyl]ethanone O-(2-hydroxyethyl)oxime, 133 mg of 2-hydroxy-5-trifluoromethylpyridine and 355 mg of triphenylphosphine are introduced together as an initial charge in 15 ml of absolute tetrahydrofuran. 236 mg of DEAD are added dropwise as a solution in 1 ml of tetrahydrofuran. The reaction mixture is stirred at RT overnight. It is evaporated to dryness and the crude product is purified via HPLC. This gives 6 mg of 1-[3-chloro-5-(3,3-dichloroallyloxy)-2-methoxyphenyl]ethanone O-[2-(3-chloro-5-trifluoromethylpyridin-2-yloxy)ethyl]oxime.

M+(ES+)=(547, 80)

¹H-NMR (CDCl₃): 2.20 (s, 3H, CH₃); 3.74 (s, 3H, OCH₃); 4.56 (dd, J=4.6; J=5.1; 2H, CH₂); 4.61 (d, J=6.3 Hz, CH₂CHCCl₂); 4.75 (dd, J=4.6; J=5.1; 2H, CH₂); 6.12 (t, J=6.3 Hz, 1H, CH₂CHCCl₂); 6.77 (d, J=3.1 Hz, 1H, aryl); 6.94 (d, J=3.1 Hz, 1H, aryl); 7.85 (d, J=2.1 Hz; 1H, pyridyl); 8.31 (d, J=1.2 Hz, 1H, pyridyl).

In analogy to the instructions for 1-[3-chloro-5-(3,3-dichloroallyloxy)-2-methoxyphenyl]-ethanone O-[2-(3-chloro-5-trifluoromethylpyridin-2-yloxy)ethyl]oxime (20) the following compounds of the general formula I were synthesized:

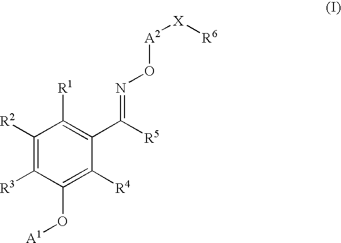

TABLE 1
for X = O

| Name | A¹ | R¹ | R² | R³ | R⁴ | R⁵ | A² | R⁶ | Physical data |
|---|---|---|---|---|---|---|---|---|---|
| (21): | CH₂CHCCl₂ | OCH₃ | Cl | H | H | CH₃ | (CH₂)₂ | 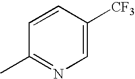 | (M + 513, 10) |
| (22): | CH₂CHCCl₂ | OCH₃ | Cl | H | H | CH₃ | (CH₂)₄ | 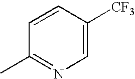 | (M + 541, 89) |
| (23): | CH₂CHCCl₂ | OCH₃ | Cl | H | H | CH₃ | (CH₂)₄ | 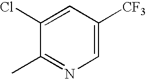 | (M + 575, 63) |
| (24): | CH₂CHCCl₂ | OCH₃ | Cl | H | H | CH₃ | (CH₂)₄ | 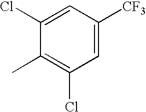 | (M + 608, 32); ¹H-NMR: 1.96- 2.06 (m, 4H, (CH2)2), 2.22 (s, 3H, CH3); 3.75 (s, 3H, OCH3); 4.09-4.14 (m, 2H); 4.21-4.29 (m, 2H); 4.61 (d, J = 6.3 Hz, 2H, OCH2CHCCl2); 6.12 (t, J = 6.3, 1H, OCH2CHCCl2); 6.77 (d, J = 3.1 Hz, 1H, aryl), 6.93 (d, J = 3.1 Hz, 1H, aryl); 7.57 (s, 2H, aryl) |

Variant for the preparation of the compound (27):

The synthesis of the amino ether side chains is described in DE 10301519 A1.

### (25): 3-{[5-(Trifluoromethyl)pyridin-2-yl]oxy}propan-1-ol

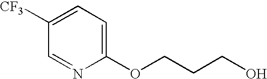

A suspension of 4 g of sodium hydride in 100 ml of DMF is admixed with 12.6 g of 1,3-propanediol. After the evolution of gas has ended the mixture is stirred at room temperature for approximately 10 minutes. Subsequently 10 g of 2-chloro-5-trifluoromethylpyridine are added as a solution in 10 ml of DMF and the reaction mixture is stirred at room temperature overnight. It is poured into water and extracted three times with dichloromethane. The combined organic phases are dried over sodium sulphate and filtered and the filtrate is evaporated to dryness. The crude product is taken up in toluene, washed with water, dried over sodium sulphate and filtered and the filtrate is evaporated to dryness.

The oily residue is stirred together with pentane and placed in a deep-freeze for crystallization. The precipitated solid is filtered off on a frit, washed with pentane and dried under reduced pressure. This gives 5.2 g of 3-{[5-(trifluoromethyl)pyridin-2-yl]oxy}propan-1-ol.

M+(ES+)=(222, 100)

¹H-NMR (CDCl₃): 2.02 (dt, 2H, CH₂); 3.75 (t, J=6 Hz, 2H, CH₂); 4.55 (t, J=6 Hz, 2H, CH₂); 6.82 (d, J=8.8 Hz, 1H, CH, pyridyl); 7.78 (dd, J=8.8 Hz, J=2.5 Hz, 1H, CH, pyridyl); 8.42 (s, 1H, CH, pyridyl).

### (26): 1-(3-{[5-(Trifluoromethyl)pyridin-2-yl]oxy}propoxy)pyrrolidine-2,5-dione

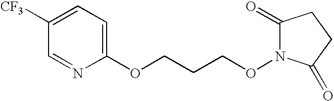

Under nitrogen 2.5 g of 3-{[5-(trifluoromethyl)pyridin-2-yl]oxy}propan-1-ol together with 1.3 g of N-hydroxyphthalimide and 2.96 g of triphenylphosphine are stirred in 200 ml of absolute tetrahydrofuran. At 0° C. 1.97 g of diethyl azodicarboxylate are added dropwise and the reaction mixture is stirred at room temperature overnight. It is evaporated to dryness and the residue is recrystallized from 4:1 hexane/EE. The solid obtained is redissolved in hot isopropanol and placed in a refrigerator for crystallization. This gives 3.25 g of 1-(3-{[5(trifluoromethyl)pyridin-2-yl]oxy}propoxy)pyrrolidine-2,5-dione as a colourless solid.

M+(ES+)=(319, 100)

¹H-NMR (CDCl₃): 2.10 (dt, 2H, CH2); 3.13 (s, 4H, CH2); 4.16 (t, J=6.3 Hz, 2H, CH2); 4.51 (t, J=6.4 Hz, 2H, CH2); 6.99 (d, J=8.7 Hz, 1H, CH, pyridyl); 8.01 (dd, J=8.8 Hz, J=6.5 Hz, 1H, CH pyridyl); 8.54 (s, 1H, CH, pyridyl)

### (27): 2-[3-(Aminooxy)propoxy]-5-(trifluoromethyl)pyridine

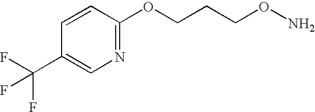

First 2 g of 1-(3-{[5-(trifluoromethyl)pyridin-2-yl]oxy}propoxy)pyrrolidine-2,5-dione are introduced as an initial charge in 60 ml of dichloromethane and 3 ml of methanol. Subsequently 378 mg of hydrazine hydrate are added. The mixture is stirred under reflux for approximately 4 h.

After the batch has cooled it is extracted with 5N ammonia solution. The organic phase is separated off, dried over sodium sulphate and filtered and the filtrate is evaporated to dryness. This gives 0.77 g of 2-[3-(aminooxy)propoxy]-5-(trifluoromethyl)pyridine.

¹H-NMR (CD₃CN): 2.02 (dt, 2H, CH₂); 3.72 (t, J=6.3 Hz, 2H, CH₂); 4.40 (t, J=6.6 Hz, 2H, CH₂); 6.88 (d, J=8.8 Hz, 1H, CH, pyridyl); 7.22 (bs, 2H, NH₂); 7.90 (dd, J=8.8 Hz, J=2.6 Hz, 1H, CH, pyridyl); 8.49 (s, 1H, CH, pyridyl).

### (28): 1-[3-Chloro-5-(3,3-dichloroallyloxy)-2-methoxyphenyl]ethanone O-[1-(6-chloropyridin-3-yl)-ethyl]oxime

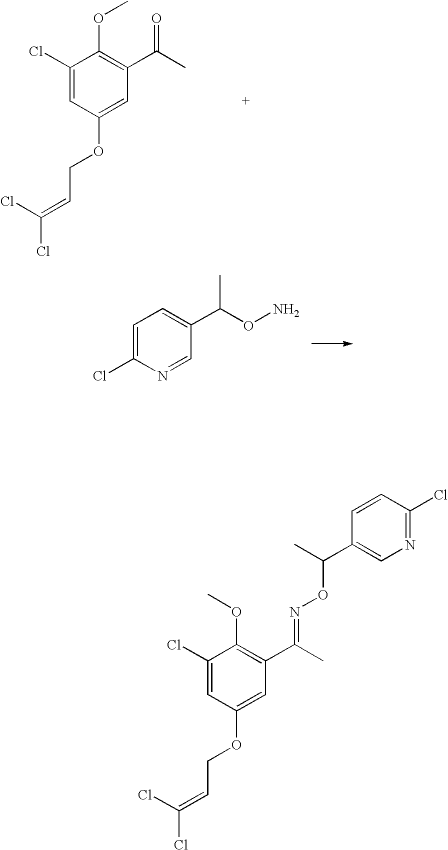

First 112 mg of O-[1-(6-chloropyridin-3-yl)ethyl]hydroxylamine are added under nitrogen to a solution of 1-[3-chloro-5-(3,3-dichloroallyloxy)-2-hydroxyphenyl]ethanone in 5 ml of absolute pyridine. The reaction mixture is stirred at 25° C. overnight. It is evaporated to dryness and the residue is chromatographed over silica gel using 4:1 cyclohexane:ethyl acetate. This gives 81 mg of 1-[3-chloro-5-(3,3-dichloroallyloxy)-2-methoxyphenyl]ethanone O-[1-(6-chloropyridin-3-yl)ethyl]oxime.

M+(ES+)=(M+463, 75)

¹H-NMR (CD₃CN): 1.58 (d, J=6.7 Hz, 3H, CH₃); 2.22 (s, 3H, CH₃); 3.58 (s, 3H, OCH₃); 4.61 (d, J=6.4 Hz, 2H, OCH₂CHCCl₂); 5.36 (g, J=6.7 Hz, 1H, CH); 6.22 (t, J=6.4 Hz, OCH₂CHCCl₂); 6.68 (d, J=3.1 Hz, 1H, CH, aryl); 7.01 (d, J=3.1 Hz, 1H, CH, aryl); 7.38 (d, J=8.3 Hz, 1H, pyridyl); 7.75 (dd, J=8.3 and 2.4 Hz, 1H, pyridyl); 8.37 (d, J=2.4 Hz, 1H, pyridyl).

In analogy to the instructions for (35): 1-[3-chloro-5-(3,3-dichloroallyloxy)-2-methoxyphenyl]ethanone O-[1-(6-chloro-pyridin-3-yl)ethyl]oxime the following compounds of the general formula I were synthesized:

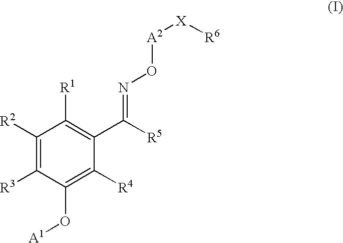

TABLE 2a
for X = direct bond

| Name | A¹ | R¹ | R² | R³ | R⁴ | R⁵ | R⁶ | A² | Physical data |
|---|---|---|---|---|---|---|---|---|---|
| (29): | CH₂CHCCl₂ | OCH₃ | Cl | H | H | CH₃ | 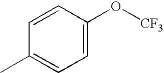 | CH₂ | (M + 498, 100) |
| (30): | CH₂CHCCl₂ | OCH₃ | Cl | H | H | CH₃ | 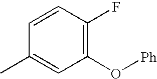 | CH₂ | (M + 524, 100) |
| (31): | CH₂CHCCl₂ | OCH₃ | Cl | H | H | cyclo- propyl | 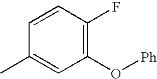 | CH₂ | (M + 550, 100) |
| (32): | CH₂CHCCl₂ | OCH₃ | Cl | H | H | CH₃ | 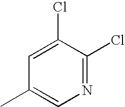 | CH₂ | (M + 483, 68) |
| (33): | CH₂CHCCl₂ | OCH₃ | Cl | H | H | CH₃ | 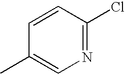 | CH₂ | (M + 449, 84) |
| (34): | CH₂CHCCl₂ | OCH₃ | Cl | H | H | CH₃ | 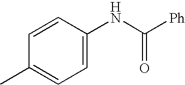 | (CH₂)₂ | (M + 547, 100) |
| (35): | CH₂CHCCl₂ | OCH₃ | Cl | H | H | CH₃ | 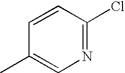 | CH(CH₃) | (M + 463, 75); ¹H-NMR: 1.58 (d, J = 6.7 Hz, 3H, CH₃); 2.22 (s, 3H, CH₃); 3.58 (s, 3H, OCH₃); 4.61 (d, J = 6.4 Hz, 2H, OCH₂CHCCl₂); 5.36 (g, J = 6.7 Hz, 1H, CH); 6.22 (t, J = 6.4 Hz, OCH₂CHCCl₂); 6.68 (d, J = 3.1 Hz, 1H, CH, aryl); 7.01 (d, J = 3.1 Hz, 1H, CH, aryl); 7.38 (d, J = 8.3 Hz, 1H, pyridyl); 7.75 (dd, J = 8.3 and 2.4 Hz, 1H, pyridyl); 8.37 (d, J = 2.4 Hz, 1H, pyridyl). |
| (36): | CH₂CHCCl₂ | OCH₃ | Cl | H | H | CH₃ | 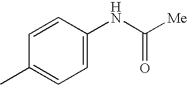 | (CH₂)₂ | (M + 485, 100) |
| (37): | CH₂CHCCl₂ | OCH₃ | Cl | H | H | CH₃ | 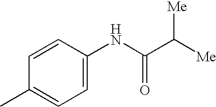 | (CH₂)₂ | (M + 513, 100) |
| (38): | CH₂CHCCl₂ | OCH₃ | Cl | H | H | CH₃ | 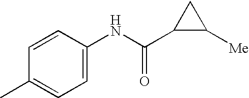 | (CH₂)₂ | (M + 525, 100) |
| (39): | CH₂CHCCl₂ | OCH₃ | Cl | H | H | CH₃ | 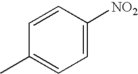 | (CH₂)₂ | (M + 473, 95) |
| (40): | CH₂CHCCl₂ | OCH₃ | Cl | H | H | CH₃ | 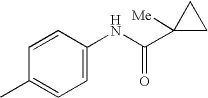 | (CH₂)₂ | (M + 525, 98) |
| (41): | CH₂CHCCl₂ | OCH₃ | Cl | H | H | CH₃ | 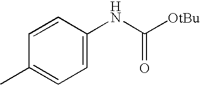 | (CH₂)₂ | (M + 543, 100) |
| (42): | CH₂CHCCl₂ | OCH₃ | Cl | H | H | CH₃ | 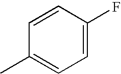 | (CH₂)₂ | (M + 460, 95) |
| (43): | CH₂CHCCl₂ | OCH₃ | Cl | H | H | CH₃ | 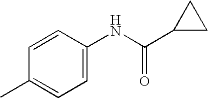 | (CH₂)₂ | (M + 511, 100) |
| (44): | CH₂CHCCl₂ | OCH₃ | Cl | H | H | CH₃ | 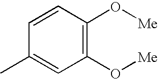 | (CH₂)₂ | (M + 489, 95) |
| (45): | CH₂CHCCl₂ | OCH₃ | Cl | H | H | CH₃ | Ph | CHCH₃ | (M + 430, 100) |
| (46): | CH₂CHCCl₂ | OCH₃ | Cl | H | H | CH₃ | 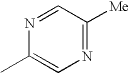 | CH₂ | (M + 430, 100) |
| (47): | CH₂CHCCl₂ | OCH₃ | Cl | H | H | CH₃ | 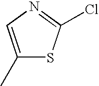 | CHCH₃ | (M + 471, 100) |
| (48): | CH₂CHCCl₂ | OCH₃ | Cl | H | H | CH₃ | 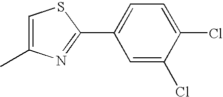 | CH₂ | (M + 567, 100) |

Ph = phenyl,
Me = methyl

In analogy to the instructions for (35): 1-[3-chloro-5-(3,3-dichloroallyloxy)-2-methoxyphenyl]ethanone O-[1-(6-chloropyridin-3-yl)ethyl]oxime the following compound of the general formula I was synthesized:

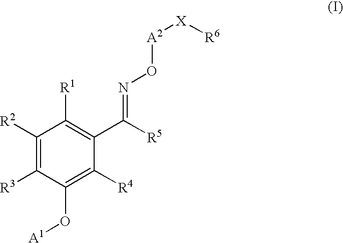

TABLE 2b
for X = O bond

| Name | A1 | R1 | R2 | R3 | R4 | R5 | R6 | A2 | Physical data |
|---|---|---|---|---|---|---|---|---|---|
| (49): | CH₂CHCCl₂ | OCH₃ | Cl | H | H | CH₃ | 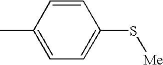 | 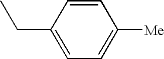 | (M + 552, 100) |

### (50): N-{4-[2-({[(1E)-{3-Chloro-5-[(3,3-dichloroprop-2-en-1-yl)oxy]-2-methoxyphenyl}(cyclopropyl)methylene]amino}oxy)ethyl]phenyl}-2-methylpropanamide

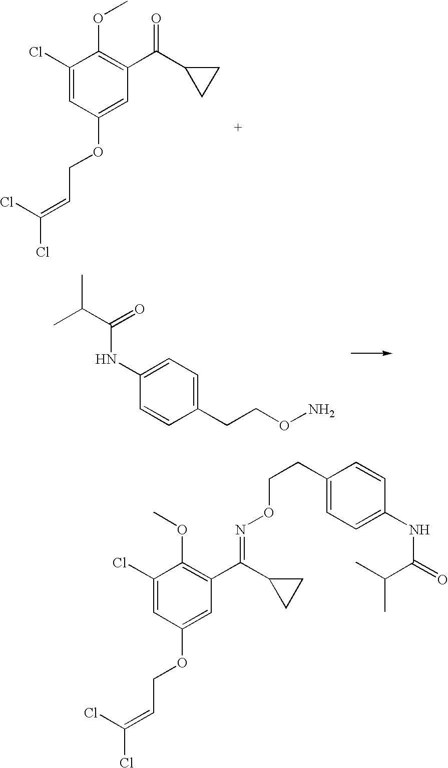

First 132 mg of N-{4-[2-(aminooxy)ethyl]phenyl}-2-methylpropanamide are added under nitrogen to a solution of 100 mg of [3-chloro-5-(3,3-dichloroallyloxy)-2-methoxyphenyl]cyclopropylmethanone in 5 ml of absolute pyridine and 5 ml of toluene. The reaction mixture is stirred under reflux overnight with a water separator. The reaction mixture is evaporated to dryness. Subsequently a further 5 ml of pyridine and toluene are added. The mixture is again stirred under reflux overnight with a water separator.

The reaction mixture is evaporated to dryness and the residue is purified by flash chromatography (4:1 cyclohexane:ethyl acetate). This gives 44.5 mg of N-{4-[2-({[(1E)-{3-chloro-5-[(3,3-dichloro-prop-2-en-1-yl)oxy]-2-methoxyphenyl}(cyclopropyl)methylene]amino}oxy)ethyl]phenyl}-2-methylpropanamide as an E/Z isomer mixture.

M+(ES+)=(539, 100)

¹H-NMR (CD₃CN): 0.41-0.43 (m, 2H, CH₂, cPr); 0.64-0.69 (m, 2H, CH₂, cPr); 0.77-0.84 (m, 4H, CH₂, cPr); 1.71-1.80 (m, 1H, CH, cPr); 2.31-2.38 (m, 1H, CH); 2.50-2.52 (m, 1H, CH); 2.80 (t, J=6.6 Hz, 2H, CH₂); 2.95 (t, J=6.7 Hz, 2H, CH₂); 3.62 (s, 3H, OCH₃); 3.76 (s, 3H, OCH₃); 4.11 (t, J=6.6 Hz, 2H, CH₂); 4.28 (t, J=6.7 Hz, 2H, CH₂); 4.59 (d, J=6.5 Hz, 2H, OCH₂CHCCl₂); 4.64 (d, J=6.4 Hz, 2H, OCH₂CHCCl₂);

6.22-6.25 (m, 2×1H, OCH₂CHCCl); 6.44 (d, J=3.0 Hz, 1H, aryl); 6.58 (d, J=3.0 Hz, 1H, aryl); 6.99-7.04 (m, H, aryl); 7.21 (d, J=8.5 Hz, 1H, aryl); 7.40 (d, J=8.5 Hz, 1H, aryl); 7.49 (d, J=8.5 Hz, 1H, aryl); 8.1-8.2 (bd, 2H, NH).

In analogy to the instructions for (50): N-{4-[2-({[(1E)-{3-chloro-5-[(3,3-dichloroprop-2-en-1-yl)oxy]-2-methoxyphenyl}(cyclopropyl)methylene]amino}oxy)ethyl]phenyl}-2-methylpropanamide the following compounds of the general formula I were synthesized:

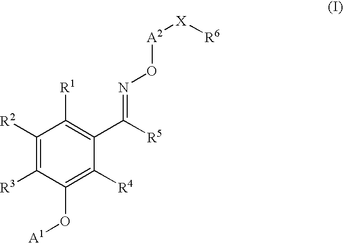

TABLE 3a
for X = direct bond

| Name | A¹ | R¹ | R² | R³ | R⁴ | R⁵ | R⁶ | A² | Physical data |
|---|---|---|---|---|---|---|---|---|---|
| (51): | CH₂CHCCl₂ | OCH₃ | Cl | H | H | IPr | 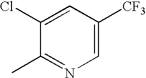 | 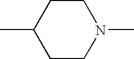 | (M + 616, 100) |
| (52): | CH₂CHCCl₂ | OCH₃ | Cl | H | H | CH₃ | 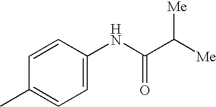 | (CH₂)₂ | (M + 539, 100) |
| (53): | CH₂CHCCl₂ | OCH₃ | Cl | H | H | cPr | 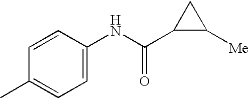 | (CH₂)₂ | (M + 551, 70) |
| (54): | CH₂CHCCl₂ | OCH₃ | Cl | H | H | cPr | 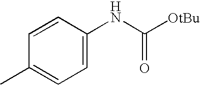 | (CH₂)₂ | (M + 569, 100) |
| (55): | CH₂CHCCl₂ | OCH₃ | Cl | H | H | cPr | 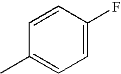 | (CH₂)₃ | (M + 486, 100) |
| (56): | CH₂CHCCl₂ | OCH₃ | Cl | H | H | cPr | 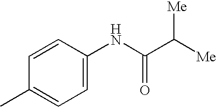 | (CH₂)₂ | (M + 581, 100) |
| (57): | CH₂CHCCl₂ | OCH₃ | Cl | H | H | CH₃ | 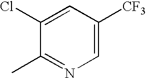 | 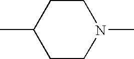 | (M + 586, 100) |
| (58): | CH₂CHCCl₂ | OCH₃ | Cl | H | H | cHexyl | 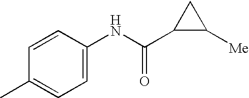 | (CH₂)₂ | (M + 593, 100) |
| (59): | CH₂CHCCl₂ | OCH₃ | Cl | H | H | IPr | 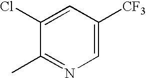 | 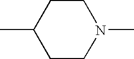 | (M + 616, 100) |
| (60): | CH₂CHCCl₂ | OCH₃ | Cl | H | H | CH(CH₃)₂ | 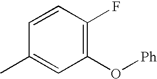 | CH₂ | (M + 552, 100) |
| (61): | CH₂CHCCl₂ | OCH₃ | Cl | H | H | CH(CH₃)₂ | 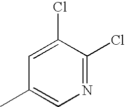 | CH₂ | (M + 512, 60) |

In analogy to the instructions for (50): N-{4-[2-({[(1E)-{3-chloro-5-[(3,3-dichloroprop-2-en-1-yl)oxy]-2-methoxyphenyl}(cyclopropyl)methylene]amino}oxy)ethyl]phenyl}-2-methylpropanamide the following compound of the general formula I was synthesized:

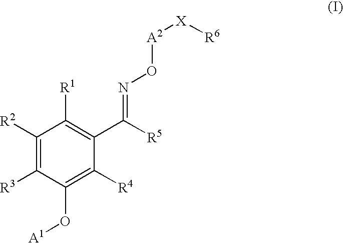

TABLE 3b
for X = O bond

| Name | A¹ | R¹ | R² | R³ | R⁴ | R⁵ | R⁶ | A² | Physical data |
|---|---|---|---|---|---|---|---|---|---|
| (62): | CH₂CHCCl₂ | OCH₃ | Cl | H | H | CH₃ | 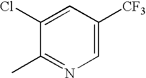 | 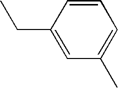 | (M + 609, 90) |

### (63): 4-{1-[3-Chloro-5-(3,3-dichloroallyloxy)-2-methoxyphenyl]ethylideneaminooxy}butyric acid

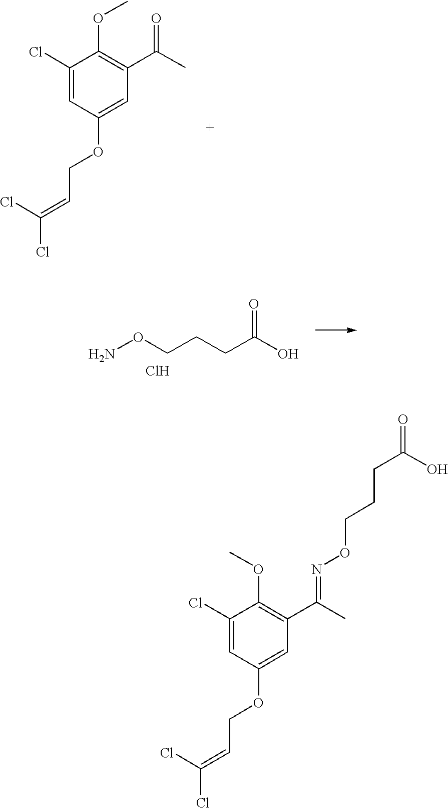

First 1.2 g of 1-[3-chloro-5-(3,3-dichloroallyloxy)-2-hydroxyphenyl]ethanone are stirred together with 664 mg of 4-aminooxybutyroyl hydrochloride at RT for 3 days in the presence of molecular sieve in 60 ml of absolute methanol. The reaction mixture is filtered, the filtrate is evaporated to dryness, the residue is diluted with ethyl acetate, washed with water, dried over sodium sulphate and filtered and the filtrate is concentrated to dryness.

The residue is taken up in methanol, 2 ml of water and 1 g of potassium carbonate are added and the reaction mixture is stirred at RT overnight. It is evaporated to dryness and the residue is taken up in 1:1 water/EE and extracted once with ethyl acetate. The aqueous phase is acidified using 1N HCl and extracted a number of times with ethyl acetate. The combined organic phases are dried over sodium sulphate and filtered and the filtrate is evaporated to dryness. This gives 354 mg of 4-{1-[3-chloro-5-(3,3-dichloroallyloxy)-2-methoxyphenyl]ethylideneaminooxy}butyric acid.

M+(ES+)=(408, 98)

¹H-NMR (CD₃CN): 2.08-2.26 (m, 2H, CH₂ and water); 2.16 (s, 3H, CH₃); 2.40 (t, J=7.3 Hz, 2H, CH₂); 3.72 (s, 3H, CH₃); 4.16 (t, J=6.3 Hz, 2H, CH₂); 4.65 (d, J=6.4 Hz, 2H, OCH₂CHCCl₂); 6.26 (t, J=6.4 Hz, 1H, OCH₂CHCCl₂); 6.81 (d, J=3.1 Hz, CH, aryl); 7.04 (d, (d, J=3.1 Hz, CH, aryl)

### (64): 4-{1-[3-Chloro-5-(3,3-dichloroallyloxy)-2-methoxyphenyl]ethylideneaminooxy}-N-phenylbutyramide

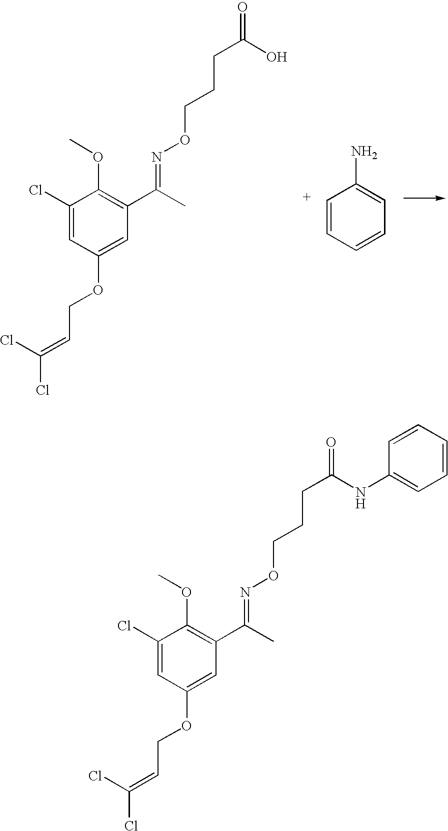

First 50 mg of 4-{1-[3-chloro-5-(3,3-dichloroallyloxy)-2-methoxyphenyl]ethylideneaminooxy}-butyric acid are introduced as an initial charge in 2 ml of dichloromethane and 13 mg of aniline, 7.5 mg of DMAP and 37 mg of triethylamine are added. Subsequently 28 mg of N-(3-dimethylaminopropyl)-N-ethylcarbodiimide hydrochloride, in solution in 1 ml of dichloromethane, are added dropwise and the reaction mixture is stirred at RT overnight. It is washed with 1N HCl, the organic phase is dried over sodium sulphate and filtered and the filtrate is evaporated to dryness. The crude product is purified by flash chromatography (eluent: 98:2 dichloromethane/methanol). This gives 18 mg of 4-{1-[3-chloro-5-(3,3-dichloroallyloxy)-2-methoxyphenyl]ethylideneaminooxy}-N-phenylbutyramide.

(M+485, 100)

¹H-NMR (CD₃CN): 2.04 (t, J=7.2 Hz, CH₂); 2.15 (s, 33.71H, CH₃); 2.44 (t, J=7.3 Hz, 2H, CH₂); 3.71 (s, 3H, OCH₃); 4.21 (t, J=6.3 Hz, 2H, CH₂); 4.64 (d, J=6.4 Hz, 2H, OCH₂CHCCl₂); 6.25 (t, J=6.4 Hz, 1H, OCH₂CHCCl₂); 6.82 (d, J=3.1 Hz, 1H, aryl); 7.04 (d, J=3.1 Hz, 1H, aryl); 7.04-7.08 (m, 1H, aniline); 7.29 (dd, 2H, J=5.8 and 6.6 Hz, aniline); 7.55 (d, 2H; J=7.6 Hz); 8.3 (bs, 1H, NH).

In the same way as for (64) (65) was synthesized:

| Name | A¹ | R¹ | R² | R³ | R⁴ | R⁵ | R⁶ | A² | Physical data |
|---|---|---|---|---|---|---|---|---|---|
| (65): | CH₂CHCCl₂ | OCH₃ | Cl | H | H | CH₃ | 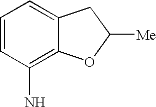 | (CH₂)₃CO | (M + 543, 100) |

### (66): Ethyl (2E)-3-{4-[(4-{[((1E)-1-{3-chloro-5-[(3,3-dichloroprop-2-en-1-yl)oxy]-2-methoxyphenyl}ethylidene)amino]oxy}butanoyl)amino]phenyl}acrylate

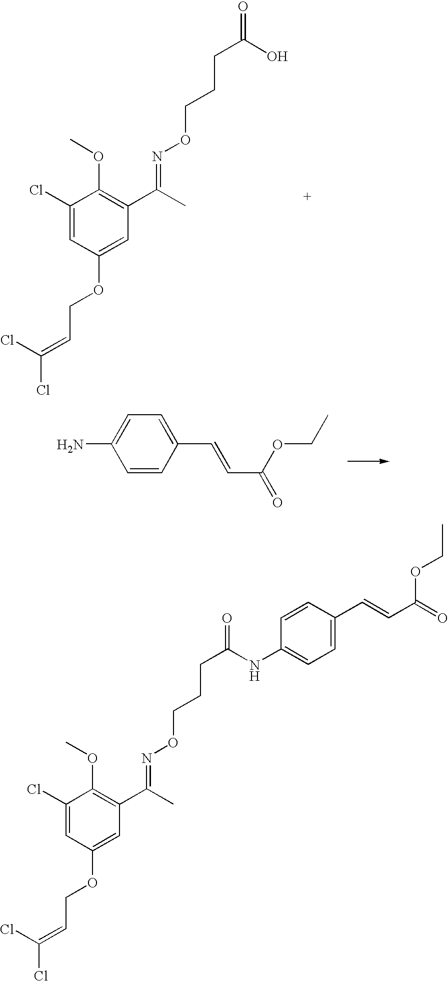

First 50 mg of 4-{1-[3-chloro-5-(3,3-dichloroallyloxy)-2-methoxyphenyl]ethylideneaminooxy}-butyric acid are introduced as an initial charge in 2 ml of dichloromethane and 17 mg of oxalyl chloride and one drop of DMF are added. After evolution of gas has ended, 25 mg of ethyl (2E)-3-(4-aminophenyl)acrylate are added dropwise as a solution in 1 ml of dichloromethane together with 37 mg of triethylamine. The mixture is stirred at room temperature overnight.

The reaction mixture is washed with 1N HCl, the organic phase is dried over sodium sulphate and filtered and the filtrate is evaporated to dryness. The crude product is purified by flash chromatography (eluent: 98:2 dichloromethane/methanol). This gives 11 mg of ethyl (2E)-3-{4-[(4-{[((1E)-1-{3-chloro-5-[(3,3-dichloroprop-2-en-1-yl)oxy]-2-methoxyphenyl}ethylidene)amino]oxy}-butanoyl)amino]phenyl}acrylate.

¹H-NMR: 1.33 (t, J=7.1 Hz, 3H, CH₃ ethyl); 2.16 (t, J=6.5 Hz, 2H, CH₂); 2.22 (s, 3H, CH₃); 2.54 (t, J=6.9 Hz, 2H, CH₂); 3.76 (s, 3H, OCH₃); 4.27-4.30 (m, 4H, 2×CH₂); 4.59 (d, J=6.3 Hz, 2H, OCH₂CHCCl₂); 6.10 (t, J=6.3 Hz, 1H, OCH₂CHCCl₂); 6.35 (d, J=16 Hz, 1H, CH═CH); 6.75 (d, J=3.0 Hz, 1H, aryl); 6.95 (d, J=3.0 Hz, 1H, aryl); 7.44-7.51 (m, 4H, aryl); 7.62 (d, J=16 Hz, 1H, CH); 7.76 (bs, 1H, NH).

In analogy to the instructions for (66): ethyl (2E)-3-{4-[(4-{[((1E)-1-{3-chloro-5-[(3,3-dichloroprop-2-en-1-yl)oxy]-2-methoxyphenyl}ethylidene)amino]oxy}butanoyl)amino]-phenyl}acrylate the following compounds of the general formula I were synthesized:

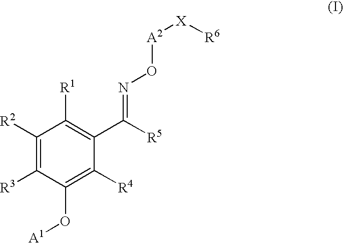

TABLE 4
for X = NH

| Name | A¹ | R¹ | R² | R³ | R⁴ | R⁵ | R⁶ | A² | Physical data |
|---|---|---|---|---|---|---|---|---|---|
| (67): | CH₂CHCCl₂ | OCH₃ | Cl | H | H | CH₃ | 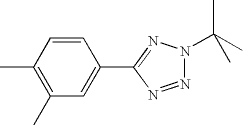 | (CH₂)₃CO | (M + 623, 100) |
| (68): | CH₂CHCCl₂ | OCH₃ | Cl | H | H | CH₃ | 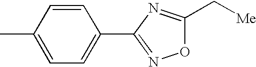 | (CH₂)₃CO | (M + 581, 100) |

### (69): 2,2-Dimethyl-2,3-dihydro-1-benzofuran-7-yl 4-{[((1E)-1-{3-chloro-5-[(3,3-dichloroprop-2-en-1-yl)oxy]-2-methoxyphenyl}ethylidene)amino]oxy}butyrate

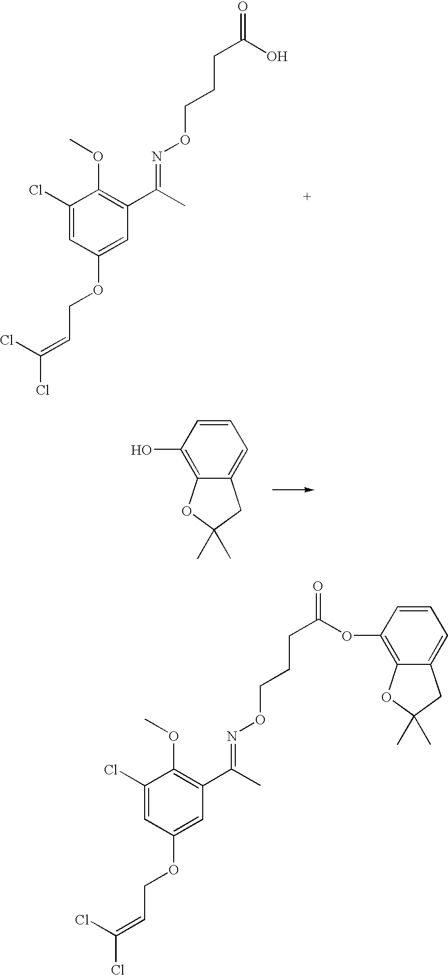

First 50 mg of 4-{1-[3-chloro-5-(3,3-dichloroallyloxy)-2-methoxyphenyl]ethylideneaminooxy}-butyric acid are introduced as an initial charge in 2 ml of dichloromethane and admixed in succession with 22 mg of 2,2-dimethyl-2,3-dihydro-1-benzofuran-7-ol, 7.5 mg of 4-dimethylaminopyridine and 40 mg of triethylamine. Subsequently 28 mg of 1,3-dicyclohexylcarbodiimide are added dropwise as a solution in 1 ml of dichloromethane and the mixture is stirred at RT overnight. It is subsequently stirred under reflux for 1 h.

The reaction mixture is washed with 1N HCl, the organic phase is dried over sodium sulphate and filtered and the filtrate is evaporated to dryness. Purification by means of HPLC gives 27 mg of 2,2-dimethyl-2,3-dihydro-1-benzofuran-7-yl 4-{[((1E)-1-{3-chloro-5-[(3,3-dichloroprop-2-en-1-yl)oxy]-2-methoxyphenyl}ethylidene)amino]oxy}butanoate as an E/Z isomer mixture.

M+(ES+)=(556, 100)

¹H-NMR (CD₃CN): 2.06-2.12 (m, 4H, 2×CH₂); 2.14 (s, 12H, 4×CH₃); 2.15 (s, 3H, CH₃); 2.18 (s, 3H, CH₃); 4.15 (t, J=6.3 Hz, 2H, CH₂); 4.25 (t, J=6.3 Hz, 2H, CH₂); 4.64-4.66 (m, 4H, 2×OCHCHCCl₂); 6.23-6.26 (m, 2H, 2×OCH₂CHCCl₂); 6.81-6.84 (m, 6H, aryl); 7.04-7.05 (m, 4H, aryl).

### (70): 2-Hydroxy-5-methoxyphenyl cyclopropyl ketone (A)

### 2,5-Dimethoxyphenyl cyclopropyl ketone (B)

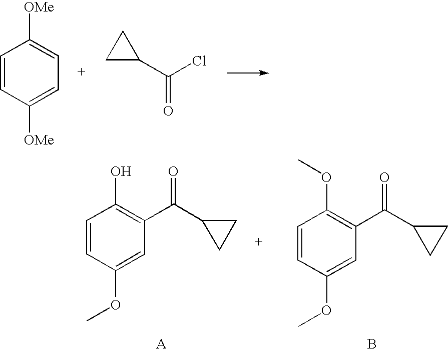

Under inert gas 48.2 g of AlCl₃ are introduced as an initial charge in 350 ml of 1,2-dichloroethane and this initial charge is admixed with 50 g of 1,4-dimethoxybenzene. Subsequently 37.8 g of cyclopropylcarbonyl chloride are slowly added dropwise and the mixture is stirred under reflux for a number of hours. After the mixture has cooled it is discharged into ice-water, a little concentrated HCl is added and the aqueous phase is extracted twice with dichloromethane. The combined organic phases are dried and concentrated. This gives 50 g of a 2:1 mixture of 2-hydroxy-5-methoxyphenyl)cyclopropyl ketone (A) and 2,5-dimethoxyphenyl cyclopropyl ketone (B) as a yellow solid which is used in the next step.

M+(ES+)=(193, 100) for A; (207, 100) for B

¹H-NMR (DMSO-D6) for A: 1.11 (m, 4H, -cPr); 3.04 (m, 1H, -cPr); 3.78 (s, 3H, —OMe); 6.92 (d, J=9 Hz, 1H, aryl); 7.17 (dd, J=2.1 Hz, 9 Hz, 1H, aryl); 7.53 (d, J=3.1 Hz, 1H, aryl); 11.58 (s, 1H, OH)

¹H-NMR (DMSO-D6) for B: 1.00 (m, 4H, -cPr); 2.71 (m, 1H, -cPr); 3.73 (s, 3H, —OMe); 3.83 (s, 3H, —OMe); 6.97 (m, 1H, aryl); 7.12 (m, 2H, aryl); 11.20 (s, 1H, OH)

### (71) 3-Chloro-2-hydroxy-5-methoxyphenyl cyclopropyl ketone-(2,5-dimethoxyphenyl cyclo-propyl ketone

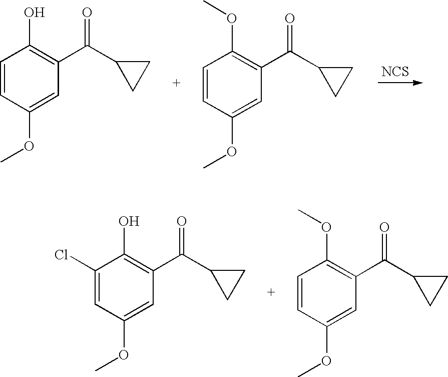

A solution of 68 g of an approximately 2:1 mixture of 1-(2-hydroxy-5-methoxyphenyl)cyclopropyl ketone and 2,5-dimethoxyphenyl cyclopropyl ketone in DMF is admixed under inert gas with 57.2 g of N-chlorosuccinimide in portions over the course of 1 h. The reaction solution undergoes brown discoloration and is stirred at RT overnight. The reaction mixture is poured into 600 ml of water, producing 53 g of 3-chloro-2-hydroxy-5-methoxyphenyl cyclopropyl ketone as a beige solid. The supernatant aqueous solution is extracted a number of times with dichloromethane, the combined organic phases are washed a number of times with water, dried over Na₂SO₄ and filtered, and the filtrate is evaporated to dryness. This gives 35 g of 2,5-dimethoxyphenyl cyclopropyl ketone as a reddish oil. Both compounds are used without purification in the next step.

M+(ES+)=(227, 100)

¹H-NMR (DMSO-D₆): 1.17 (m, 4H, -cPr); 3.07 (m, 1H, -cPr); 3.81 (s, 3H, —OMe); 7.42 (d, J=3 Hz, 1H, aryl); 7.66 (d, J=3 Hz, 1H, aryl); 12.25 (s, 1H, OH).

### (72): 3-Chloro-2,5-dihydroxyphenyl cyclopropyl ketone

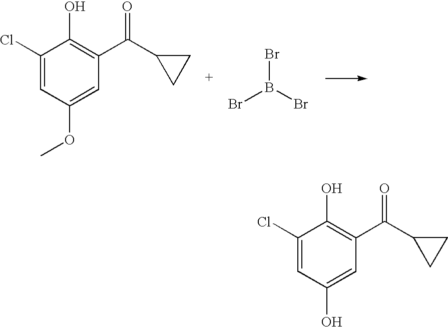

Under inert gas 53 g of 3-chloro-2-hydroxy-5-methoxyphenyl cyclopropyl ketone are introduced as an initial charge in 400 ml of dichloromethane and at 0° C. a solution of 44.2 ml of BBr₃ in 100 ml of dichloromethane is added dropwise. Subsequently the mixture is stirred at room temperature for 1 h and poured carefully into ice-water. After phase separation the aqueous phase is extracted once with dichloromethane and the combined organic phases are dried and concentrated.

M+(ES+)=(213, 100)

¹H-NMR (DMSO-D₆): 1.16 (m, 4H, -cPr); 3.13 (m, 1H, -cPr); 7.16 (d, J=2.8 Hz, 1H, aryl); 7.24 (d, J=2.8 Hz, aryl); 9.5 (s, 1H, OH); 11.8 (s, 1H, OH)

### (73): 2,5-Dihydroxyphenyl cyclopropyl ketone

### (74): 3-Chloro-2,5-dihydroxyphenyl cyclopropyl ketone

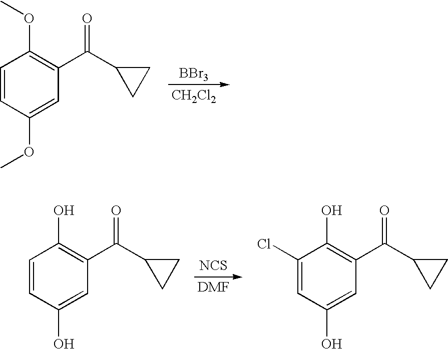

(73): Under inert gas 35 g of 2,5-dimethoxyphenyl cyclopropyl ketone are introduced as an initial charge in 300 ml of dichloromethane and cooled to 0° C. Subsequently a solution of 32 ml of BBr₃ in 100 ml of dichloromethane is slowly added dropwise. Thereafter the mixture is stirred at room temperature for 1 h and poured carefully into ice-water. After phase separation the aqueous phase is extracted once with dichloromethane and the combined organic phases are dried and concentrated. This gives 34 g of 2,5-dihydroxyphenyl cyclopropyl ketone in the form of a reddish oil which is used without further purification in the next step.

M+(ES+)=(195, 100)

(74): Over a period of 33.1 g of N-chlorosuccinimide are added under nitrogen and in portions to a solution of 34 g of 2,5-dihydroxyphenyl cyclopropyl ketone in 400 ml of DMF. The reaction solution undergoes brown discoloration and is stirred at RT overnight. The reaction mixture is poured into 0.5 l of water and extracted a number of times with dichloromethane. The combined organic phases are washed a number of times with water, dried over Na₂SO₄ and filtered and the filtrate is evaporated to dryness. This gives 39 g of product (75% purity according to LC-MS) as a solid which is used without purification in the next step.

### (75): (2-Hydroxy-5-methoxyphenyl)-2-methylpropan-1-one

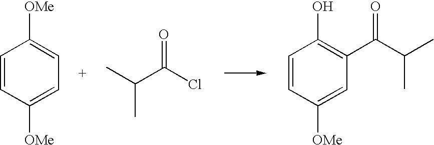

At room temperature 48.2 g of AlCl₃ and 50 g of 1,4-dimethoxybenzene are introduced as an initial charge in 350 ml of 1,2-dichloroethane and this initial charge is admixed slowly dropwise with 37.9 ml of isobutyryl chloride. The reaction mixture is subsequently stirred under reflux for 16 h. It is worked up by cooling it, pouring it cautiously with stirring onto 500 ml of ice and stirring the subsequent mixture for 10 minutes. The phases are then separated and the aqueous phase is extracted 2× with dichloromethane. Combined organic phases are dried over sodium sulphate and evaporated to dryness. This gives 77 g of mildly impure product (92% purity according to LC-MS) as an orange coloured oil which is used without purification in the next step.

M+(ES+)=(195, 100)

¹H-NMR (DMSO-D₆): 1.13 (d, J=6.8 Hz, 6H, iPr); 3.74 (m, 1H, iPr); 3.75 (s, 3H, OMe); 6.90 (d, J=9 Hz, 1H, aryl); 7.14 (dd, J=3.1 Hz, 9 Hz, aryl); 7.30 (d, J=3.1 Hz, 1H, aryl); 11.4 (s, 1H, OH)

### (76): (3-Chloro-2-hydroxy-5-methoxyphenyl)-2-methylpropan-1-one

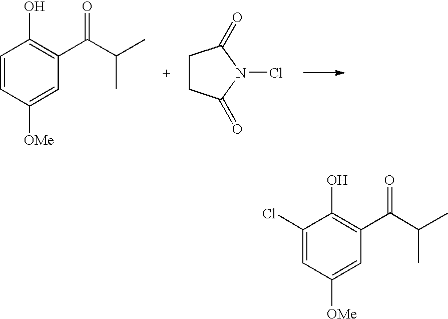

Over the course of 1 h 53.6 g of N-chlorosuccinimide are added under nitrogen and in portions to a solution of 60 g of (2-hydroxy-5-methoxyphenyl)-2-methylpropan-1-one in 600 ml of DMF. The reaction solution undergoes brown discoloration and is stirred at RT overnight. The reaction mixture is poured into 1.2 l of water and extracted a number of times with dichloromethane. The combined organic phases are washed a number of times with water, dried over Na₂SO₄ and filtered and the filtrate is evaporated to dryness. This gives 70 g of product (75% purity according to LC-MS) as a solid which is used without purification in the next step.

M+(ES+)=(229, 100)

¹H-NMR (DMSO-D₆): 1.15 (d, J=6.8 Hz, 6H, iPr); 3.79 (m, 1H, iPr); 3.79 (s, 3H, OMe); 7.40 (s, 2H, aryl); 12.0 (s, 1H, OH)

### (77): (3-Chloro-2,5-dihydroxyphenyl)-2-methylpropan-1-one

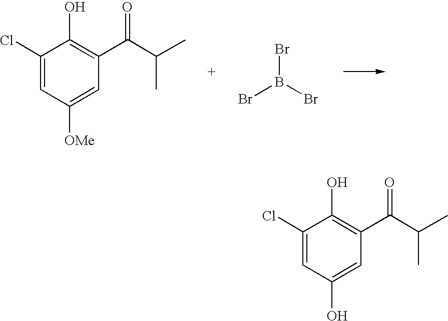

Under inert gas 36 g of (3-chloro-2-hydroxy-5-methoxyphenyl)-2-methylpropan-1-one are introduced as an initial charge in 360 ml of dichloromethane, and this initial charge is cooled to 0° C. and admixed dropwise over the course of 10 minutes with a solution of 30 ml of BBr₃ in 180 ml of dichloromethane. After 2 h of subsequent stirring at the given temperature the reaction mixture is poured onto 0.7 l of ice, stirred for 30 minutes thereafter and neutralized by addition of 350 ml of saturated NaHCO₃ solution. Following phase separation the aqueous phase is extracted 3× with 150 ml of dichloromethane and the combined organic phases are dried and evaporated to dryness. This gives 33 g of product (75% purity according to LC-MS) as an oil which is used without purification in the next step.

M+(ES+)=(215, 100)

¹H-NMR (DMSO-D₆): 1.14 (d, J=6.8 Hz, 6H, iPr); 3.62 (m, 1H, iPr); 7.16 (d, J=2.8 Hz, 1H, aryl); 7.26 (d, J=2.8 Hz, aryl); 9.5 (s, 1H, OH); 11.9 (s, 1H, OH)

### (78): 2,5-Dimethoxyphenyl cyclohexyl ketone+2-hydroxy-5-methoxyphenyl cyclohexyl ketone

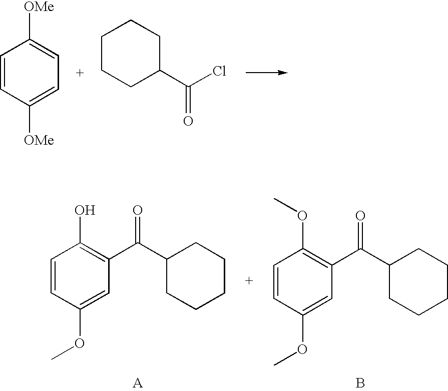

At room temperature 48.2 g of AlCl₃ and 50 g of 1,4-dimethoxybenzene are introduced as an initial charge in 350 ml of 1,2-dichloroethane and this initial charge is admixed slowly dropwise with 53 g of cyclohexylcarbonyl chloride. The reaction mixture is subsequently stirred under reflux for 16 h. For workup it is cooled and poured cautiously with stirring onto 500 ml of ice, followed by stirring for 10 minutes. Subsequently the phases are separated and the aqueous phase is extracted 2× with dichloromethane. Combined organic phases are dried over sodium sulphate and evaporated to dryness. The crude product obtained was 93 g of mildly impure product (96% purity according to LC-MS) as a mixture of 2,5-dimethoxyphenyl cyclohexyl ketone (B, 83%) and 2-hydroxy-5-methoxyphenyl cyclohexyl ketone (A, 14%) as an orange-coloured oil which is used without purification in the next step.

M+(ES+)=(235, 100) for B; (221, 100) for A

¹H-NMR (DMSO-D₆) (B only): 1.18 (m, 1H, -cHex); 1.37 (bm, 4H, -cHex); 1.65 (m, 1H, -cHex); 1.73 (m, 2H, -cHex); 1.83 (m, 2H, -cHex); 3.46 (m, 1H, -cHex); 3.76 (s, 6H, —OMe); 6.91 (d, J=9 Hz, 1H, aryl); 7.16 (dd, J=3.1 Hz, 9 Hz, 1H, aryl); 7.29 (d, J=3.1 Hz, 1H, aryl); 9.08 (s, 1H, OH); 11.39 (s, 1H, OH)

### (79): 2,5-Dihydroxyphenyl cyclohexyl ketone

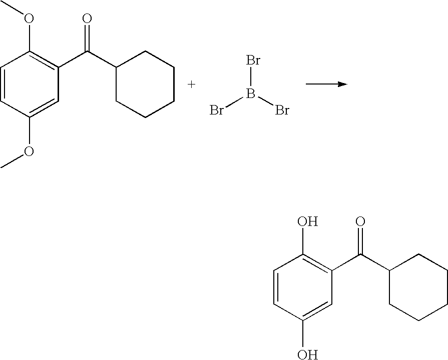

Under inert gas 84.3 g of 2,5-dihydroxyphenyl cyclohexyl ketone are introduced as an initial charge in 550 ml of dichloromethane, and this initial charge is cooled to 0° C. and admixed dropwise over a period of 30 minutes with a solution of 68 ml of BBr₃ in 200 ml of dichloromethane. After 2 h of subsequent stirring at the given temperature the reaction mixture is poured onto 0.7 l of ice, followed by subsequent stirring for 30 minutes and neutralization by addition of 400 ml of saturated NaHCO₃ solution. Following phase separation the aqueous phase is extracted a number of times with dichloromethane and the combined organic phases are dried and evaporated to dryness. This gives 37.7 g of 2,5-dihydroxyphenyl cyclohexyl ketone (99% purity according to LC-MS) as an oil which is used without purification in the next step.

M+(ES+)=(221, 100)

¹H-NMR (DMSO-D₆): 1.19 (m, 1H, -cHex); 1.35 (bm, 4H, -cHex); 1.67 (m, 1H, -cHex); 1.78 (bm, 4H, -cHex); 3.34 (m, 1H, -cHex); 6.81 (d, J=8.8 Hz, 1H, Aryl); 6.97 (dd, J=2.9 Hz, 8.8 Hz, 1H, aryl); 7.20 (d, J=2.9 Hz, 1H, aryl); 9.08 (s, 1H, OH); 11.37 (s, 1H, OH)

### (80): 3-Chloro-2,5-dihydroxyphenyl cyclohexyl ketone

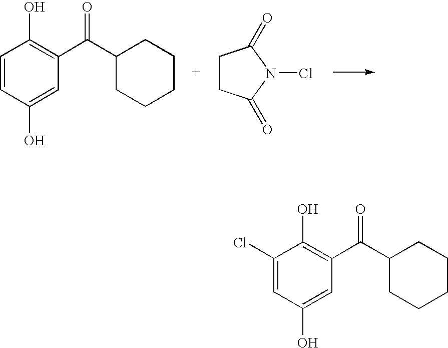

Over the course of 1 h 29 g of N-chlorosuccinimide are added under nitrogen and in portions to a solution of 36.7 g of 2,5-dihydroxyphenyl cyclohexyl ketone in 170 ml of DMF. The reaction solution undergoes brown discoloration and is stirred at RT overnight. The reaction mixture is poured into 0.6 l of water and extracted a number of times with dichloromethane. The combined organic phases are washed a number of times with water, dried over Na₂SO₄ and filtered and the filtrate is evaporated to dryness. This gives 70 g of 3-chloro-2,5-dihydroxyphenyl cyclohexyl ketone (50% purity according to LC-MS) as a solid which is used without purification in the next step.

M+(ES+)=(255, 100)

¹H-NMR (DMSO-D₆): 1.10-1.39 (bm, 5H, -cHex); 1.57-1.88 (m, 5H, -cHex); 3.35 (m, 1H, -cHex); 7.16 (d, J=2.8 Hz, 1H, aryl); 7.28 (m, J=2.8 Hz, 1H, aryl); 9.50 (s, 1H, OH); 11.96 (s, 1H, OH)

### BIOLOGICAL EXAMPLES

### Example No. A

Spodoptera frugiperda test (spray treatment)

| | |
|---|---|
| Solvent: | 78 parts by weight of acetone |
| | 1.5 parts by weight of dimethylformamide |
| Emulsifier: | 0.5 part by weight of alkylaryl polyglycol ether |

An appropriate preparation of active compound is produced by mixing 1 part by weight of active compound with the stated amounts of solvent and emulsifier and diluting the concentrate to the desired concentration using emulsifier-containing water.

Maize leaf discs (Zea mays) are sprayed with a preparation of active compound at the desired concentration and after they have dried off they are populated with caterpillars of the army worm (Spodoptera frugiperda).

After the desired time the activity is measured in %. Here 100% means that all of the caterpillars have been killed; 0% means that no caterpillar has been killed.

In this test effective activity is exhibited by, for example, the following compounds of the Preparation Examples: see table

TABLE A

| Example | Structure | g a. i./ha | % 7 d |
|---|---|---|---|
| 24 | 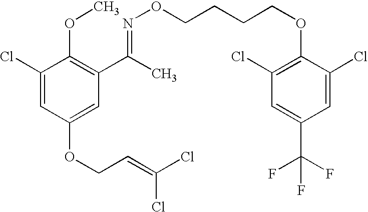 | 500 | 100 |
| 21 | 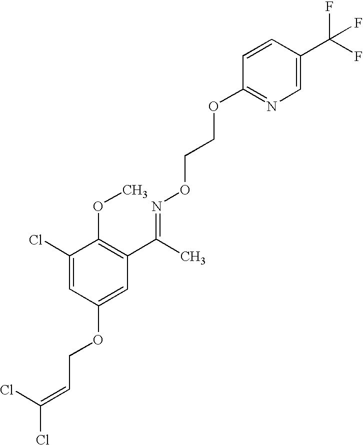 | 500 | 100 |
| 22 | 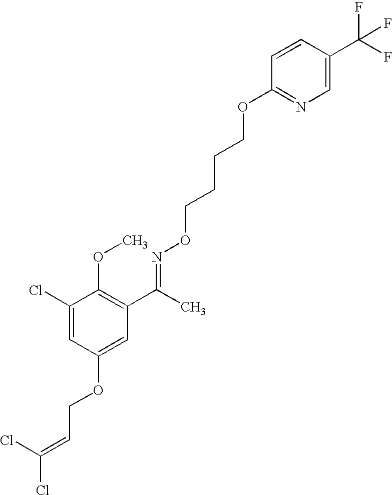 | 500 | 100 |
| 23 | 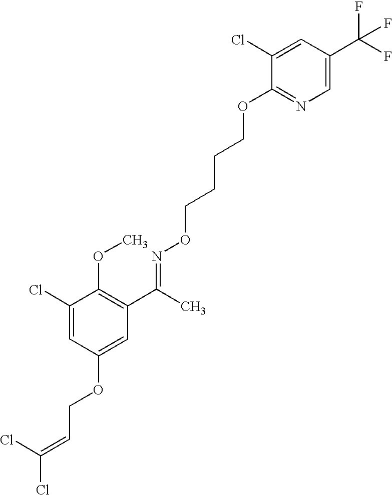 | 500 | 100 |
| 20 | 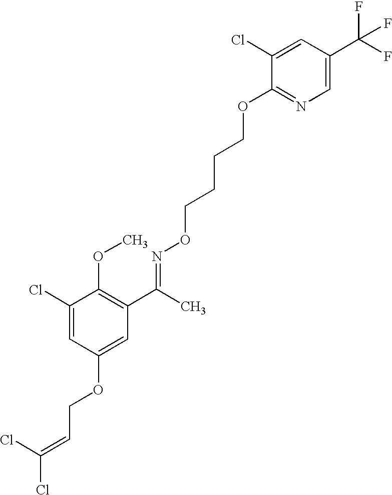 | 500 | 100 |
| 49 | 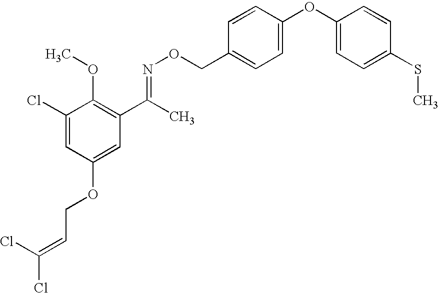 | 500 | 100 |
| 29 | 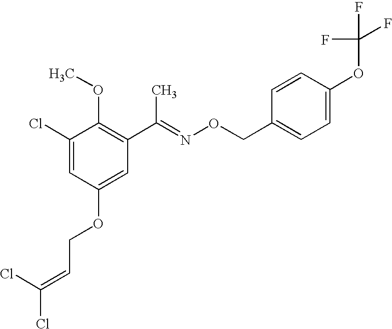 | 500 | 100 |
| 40 | 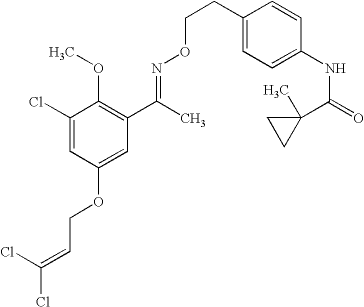 | 500 | 100 |
| 41 | 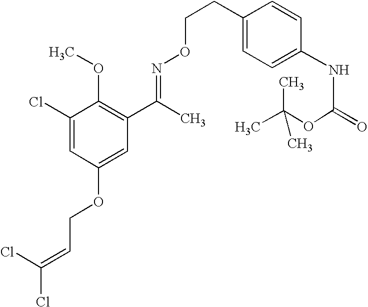 | 500 | 100 |
| 42 | 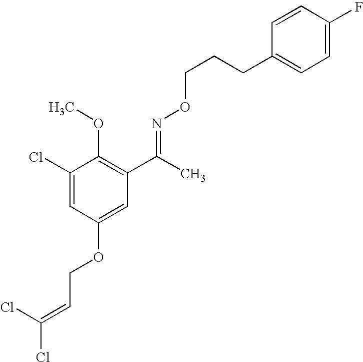 | 500 | 100 |
| 39 | 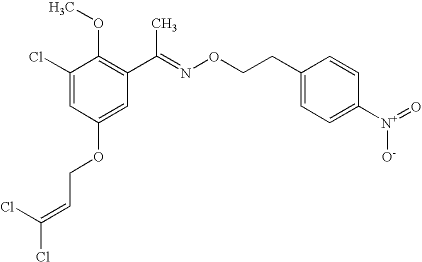 | 500 | 100 |
| 30 | 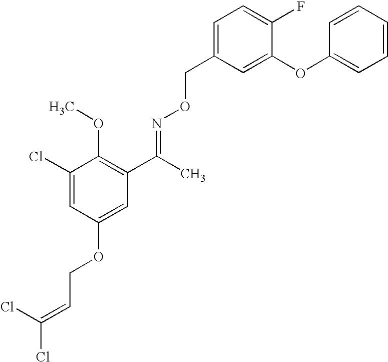 | 500 | 100 |
| 45 | 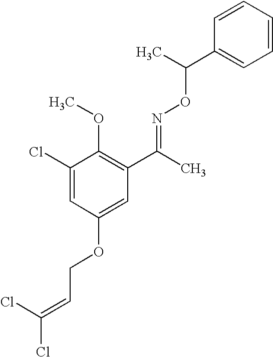 | 500 | 83 |

### Example No. B

Phaedon test (spray treatment)

| | |
|---|---|
| Solvent: | 78 parts by weight of acetone |
| | 1.5 parts by weight of dimethylformamide |
| Emulsifier: | 0.5 part by weight of alkylaryl polyglycol ether |

An appropriate preparation of active compound is produced by mixing 1 part by weight of active compound with the stated amounts of solvent and emulsifier and diluting the concentrate to the desired concentration using emulsifier-containing water.

Chinese cabbage leaves (Brassica pekinensis) are sprayed with a preparation of active compound at the desired concentration and after they have dried off they are populated with larvae of the mustard beetle (Phaedon cochleariae).

After the desired time the activity is measured in %. Here 100% means that all of the beetle larvae have been killed; 0% means that no beetle larvae have been killed.

In this test effective activity is exhibited by, for example, the following compounds of the Preparation Examples: see table

TABLE B

| Example | Structure | g a. i./ha | % 7 d |
|---|---|---|---|
| 14 | 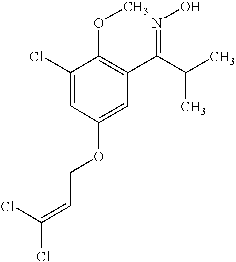 | 500 | 100 |

### Example No. C

Aedes Aegypti test

| | |
|---|---|
| Solvent: | 1000 parts by weight of dimethyl sulphoxide |

An appropriate preparation of active compound is produced by mixing the active compound with the stated amounts of solvent and diluting the concentrate to the desired concentration with water.

The Aedsae aegypti larvae (third larval stage, L3) are treated with a preparation of active compound at the desired concentration.

After the desired time the activity is measured in %. Here 100%, means that all of the Aedsae aegypti have been killed; 0% means that no Aedsae aegypti have been killed, and -1 denotes that no evaluation was possible.

In this test effective activity is exhibited by, for example, the following compound of the Preparation Examples: see table

TABLE C

| Example | Structure | g a. i./ha | % 2 d |
|---|---|---|---|
| 14 | 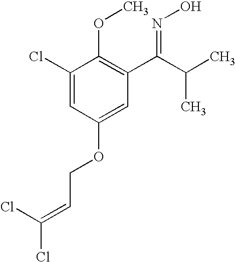 | 20 | 80 |

1. A compound of formula (I) 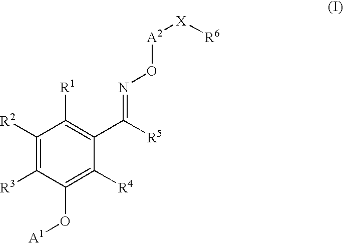
in which
A¹ is one of the moieties —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—C(halogen)₃, —(CH₂)₂—CH(halogen)₂, —CH₂—CH(halogen)-CH(halogen)₂, —CH₂—CH═CClCH₃,
A²-X is either a direct bond or a straight-chain or branched or cyclic alkanediyl or an alkenediyl having in each case up to 8 carbon atoms and which in each case, optionally in attachment to the carbon chain, contains an oxygen atom, sulfur atom or a moiety selected from SO, SO₂, NH and N(C₁-C₄-alkyl), or
A²-X is —(C₁-C₄-alkyl)-(CO)—O—, (C₁-C₄-alkyl)-(CO)—(NH)—, —(C₁-C₄-alkyl)-(CO)-[N—(C₁-C₄-alkyl)]-, —(C₁-C₄-alkyl)-(CO)—S—, —(C₁-C₄-alkyl)-O—(CO)—, (C₁-C₄-alkyl)-(NH)—(CO)—, —(C₁-C₄-alkyl)-[N—(C₁-C₄-alkyl)]-(CO)—, —(C₁-C₄-alkyl)-S—(CO)—, —(C₂-C₈-alkenyl)-(CO)—O—, —(C₂-C₈-alkenyl)-(CO)—(NH)—, —(C₂-C₈-alkenyl)-(CO)-[N—(C₁-C₄-alkyl)]-, —(C₂-C₈-alkenyl)-(CO)—S—, —(C₂-C₈-alkenyl)-O—(CO)—(C₂-C₈-alkenyl)-(NH)—(CO—)-, —(C₁-C₄-alkyl)-[N—(C₁-C₄-alkyl)]-(CO)—, —(C₂-C₈-alkenyl)-S—(CO)—, —(C₁-C₄-alkyl)-[N—(C₁-C₄-alkyl)]-(CO)—, —(C₂-C₈-alkenyl)-S—(CO)—, —(C₁-C₄-alkyl)-(SO₂)—(NH)—, —(C₂-C₈-alkenyl)-(CO)—S—, —(C₂-C₈-alkenyl)-(SO₂)—(NH)—, —(C₁-C₄-alkyl)-O—N═C(R⁹)—, —(C₁-C₈-alkenyl)-O—N═C(R⁹)—, where R⁹ is C₁-C₄-alkyl or is aryl, or
A²-X is further a carbocyclic, optionally mono- or polyunsaturated, 5-, 6- or 7-membered ring system or is an optionally mono- or polyunsaturated, 7-10-membered bicyclic ring system, which optionally may be interrupted one or more times by oxygen, sulfur, sulfonyl, sulfoxyl, carbonyl, NO, or by optionally alkyl-substituted nitrogen, and optionally may be substituted one or more times, the attachment of these ring systems to the oxime oxygen always being via carbon, and the attachment to the radical R⁶ being via carbon or a heteroatom (nitrogen or sulfur),
there being located between X and R⁶, optionally, the bridging element 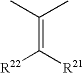
in which the radicals R²¹ and R²² independently of one another are hydrogen, nitro, hydroxyl, amino, cyano, cyanato, thiocyanato, formyl, halogen, in each case optionally cyano-, halogen- or C₁-C₆-alkoxy-substituted alkyl, alkoxy, alkylthio, alkylsulfinyl, alkylsulfonyl, alkylamino, dialkylamino or alkylcarbonylamino having in each case 1 to 4 carbon atoms in the alkyl groups, are C₁-C₄-alkyl-carbonyl, C₁-C₄-alkoxy-carbonyl, C₁-C₄-alkoximinoformyl, C₁-C₄-alkoximino-acetyl, or are C₂-C₄-alkenyl or C₂-C₄-alkynyl,
R¹ is hydrogen, nitro, hydroxyl, amino, cyano, fluoro, chloro, bromo, iodo, or is in each case optionally cyano-, fluoro-, chloro-, methoxy-, ethoxy-, n- or isopropoxy-substituted C₁-C₆-alkyl, C₃-C₆-cycloalkyl, C₃-C₆-cycloalkyloxy, C₃-C₆ alkenyl, C₃-C₆ alkenyloxy, C₃-C₆ alkynyl, C₃-C₆ alkynyloxy, C₁-C₆ alkylcarbonyloxy, C₁-C₃ alkylsulfonyl, methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino, dimethylamino, diethylamino, dipropylamino, acetylamino, propionylamino, n- or isobutyroylamino, methoximinomethyl, ethoximinomethyl, methoximinoethyl or ethoximinoethyl,
R² is hydrogen, nitro, hydroxyl, amino, cyano, cyanato, thiocyanato, formyl, halogen, is in each case optionally cyano-, halogen- or C₁-C₆-alkoxy-substituted alkyl, alkoxy, alkylthio, alkylsulfinyl, alkylsulfonyl, alkylamino, dialkylamino or alkylcarbonylamino having in each case 1 to 6 carbon atoms in the alkyl groups, is C₁-C₆-alkyl-carbonyl, C₁-C₆-alkoxy-carbonyl, C₁-C₆-alkoximinoformyl, C₁-C₆-alkoximino-acetyl, or is C₂-C₆-alkenyl or C₂-C₆-alkynyl,
R³ is hydrogen, nitro, hydroxyl, amino, cyano, halogen, is in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁴ is hydrogen, nitro, hydroxyl, amino, cyano, halogen, is in each case optionally cyano-, halogen- or C₁-C₆-alkoxy-substituted alkyl, alkoxy, alkylthio, alkylamino, dialkylamino or alkylcarbonylamino having in each case 1 to 6 carbon atoms in the alkyl groups,
R⁵ is hydrogen, straight-chain or branched or cyclic halogenalkanyl or halogenalkenyl having in each case up to 8 carbon atoms, is aryl or substituted aryl,
R⁶ is an alkenyl moiety, an alkynyl moiety having in each case 2 to 6 carbon atoms or cycloalkenyl moiety having 4 to 6 carbon atoms, which is optionally substituted by at least one substituent from the group nitro, cyano, carboxyl, carbamoyl, hydroxyl, carbonyl (C═O), hydroximino (C═N—OH), C₁-C₆-alkoxy, C₁-C₆-alkoxy-carbonyl, C₁-C₆-alkylamino, di(C₁-C₆-alkyl)amino, C₁-C₆-alkylamino-carbonyl, C₁-C₆-alkoxy-carbonylamino, C₁-C₆-alkoxy-C₁-C₆-alkoxy, C₁-C₆-alkoxyimino, C₃-C₆-alkenyloxy, C₃-C₆-alkynyloxy, C₃-C₆-alkenyloxy-carbonyl, C₃-C₆-alkynyloxy-carbonyl, C₃-C₆-alkenyloxyimino, C₃-C₆-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl, or
R⁶ is a moiety -A³-Z, where
A³ is a single bond or is straight-chain or branched and optionally halogen- or C₃-C₆-cycloalkyl-substituted alkanediyl having 1 to 6 carbon atoms and
Z is optionally nitro-, hydroxyl-, mercapto-, amino-, formyl-, cyano-, carboxyl-, carbamoyl-, halogen-, C₁-C₆-alkyl-, C₁-C₆-hydroxyalkyl-, C₁-C₆-haloalkyl-, C₁-C₆-alkyl-carbonyl-, C₁-C₆-haloalkyl-carbonyl-, C₁-C₆-alkoxy-, C₁-C₆-hydroxyalkoxy-, C₁-C₆-haloalkoxy-, C₁-C₆-alkoxy-carbonyl-, C₁-C₆-haloalkoxy-carbonyl-, C₁-C₆-alkoxy-C₁-C₆-alkyl-, C₁-C₆-haloalkoxy-C₁-C₆-alkyl-, C₁-C₂-alkylenedioxy-, C₁-C₂-haloalkylenedioxy-, C₁-C₆-alkoxyimino-C₁-C₆-alkyl-, C₂-C₆-alkenyl-, C₂-C₆-alkenyl-carbonyl-, C₂-C₆-haloalkenyl-, C₂-C₆-haloalkenyl-carbonyl-, C₂-C₆-alkenyloxy-C₁-C₆-alkyl-, C₂-C₆-haloalkenyloxy-C₁-C₆-alkyl-, C₂-C₆-alkynyl-, C₂-C₆-haloalkynyl-, C₂-C₆-alkenyloxy-, C₂-C₆-alkenyloxy-carbonyl-, C₂-C₆-haloalkenyloxy-, C₂-C₆-haloalkenyloxy-carbonyl-, C₂-C₆-alkynyloxy-, C₂-C₆-alkynyloxy-carbonyl-, C₂-C₆-haloalkynyloxy-, C₂-C₆-haloalkynyloxy-carbonyl-, C₂-C₆-alkynyloxy-C₁-C₆-alkyl-, C₂-C₆-haloalkynyloxy-C₁-C₆-alkyl-, C₃-C₈-cycloalkyl-, C₃-C₈-cycloalkyl-carbonyl-, C₃-C₈-cycloalkyl-C₁-C₆-alkyl-, C₃-C₈-cycloalkyl-C₁-C₆-alkyl-carbonyl-, C₃-C₈-cycloalkyloxy-, C₃-C₈-cycloalkyloxy-carbonyl-, C₃-C₈-cycloalkyl-C₁-C₆-alkoxy-, C₃-C₈-cycloalkyl-C₁-C₆-alkoxy-carbonyl-, C₃-C₈-cycloalkyloxy-C₁-C₆-alkyl-, C₃-C₈-cycloalkyloxy-C₁-C₆-alkoxy-, C₃-C₈-cycloalkyl-C₁-C₆-alkoxy-C₁-C₆-alkyl-, C₁-C₆-alkyl-carbonyl-C₁-C₆-alkyl-, C₁-C₆-alkoxy-carbonyl-C₁-C₆-alkyl-, C₁-C₆-alkylthio-, C₁-C₆-haloalkylthio-, C₁-C₆-alkylsulfinyl-, C₁-C₆-haloalkylsulfinyl-, C₁-C₆-alkylsulfonyl-, C₁-C₆-haloalkylsulfonyl-, C₂-C₆-alkenylthio-, C₂-C₆-haloalkenylthio-, C₂-C₆-alkynylthio-, C₃-C₆-cycloalkylthio-, C₃-C₆-cycloalkyl-C₁-C₆-alkylthio-, C₁-C₆-alkylamino-, C₁-C₆-alkylamino-carbonyl-, di(C₁-C₆-alkyl)amino-, di(C₁-C₆-alkyl)amino-carbonyl-, C₁-C₆-alkyl-carbonylamino-, C₁-C₆-haloalkyl-carbonylamino-, C₁-C₆-alkoxy-carbonylamino-, C₁-C₆-alkyl-aminocarbonylamino-substituted, or phenyl-, phenyloxy-, benzyl-, benzyloxy-, phenylamino- or benzylamino-substituted (where in each case the phenyl groups are optionally substituted by nitro, hydroxyl, mercapto, amino, cyano, C₁-C₆-alkyl, C₁-C₆-haloalkyl, C₁-C₆-alkyl-carbonyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-haloalkenyloxy, C₂-C₆-alkynyloxy, C₁-C₆-alkylthio, C₁-C₆-alkylsulfinyl, C₁-C₆-alkylsulfonyl or C₁-C₆-alkoxy-carbonyl) aryl or monocyclic or bicyclic heteroaryl having up to 10 carbon atoms and at least one heteroatom from the group N (nitrogen, 1 to 5 N atoms), O (oxygen, 1 or 20 atoms), sulfur (1 or 2 S atoms) and optionally, as a replacement or in addition, an SO or SO₂ moiety, and optionally in addition a carbonyl moiety (C═O) and/or a thiocarbonyl moiety (C═S) as part of the heterocycle, or
R⁶ is one of the following moieties 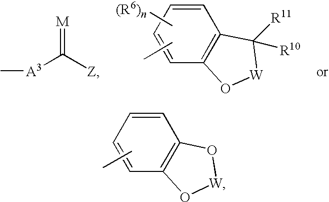
in which
A³ is a single bond or is a C₁-C₆ alkylene bridge which is optionally substituted one or more times by halogen or by C₃-C₈ cycloalkyl,
M is oxygen or NOR⁷, where
R⁷ is hydrogen, C₁-C₁₂alkyl, C₃-C₈cycloalkyl, C₁-C₆alkylcarbonyl, C₂-C₆-alkenyl, C₂-C₆alkynyl, aryl, heterocyclyl or benzyl, with alkyl, cycloalkyl, alkenyl and alkynyl radicals being unsubstituted or being optionally substituted one to five times by a radical from the following group: halogen, —N₃, CN, NO₂, OH, SH, C₁-C₆alkoxy, C₁-C₆haloalkoxy, C₂-C₆alkenyloxy, C₂-C₆-haloalkenyloxy, C₃-C₆-alkynyloxy, C₃-C₆haloalkynyloxy, C₃-C₈cycloalkyl-C₁-C₆alkoxy, C₁-C₆-alkylcarbonyl, C₁-C₆haloalkylcarbonyl, C₁-C₆alkoxycarbonyl, C₁-C₆alkylcarbonyl-C₁-C₆alkyl, C₁-C₆alkoxycarbonyl-C₁-C₆alkyl, C₁-C₆alkylthio, C₂-C₆alkylthio, C₃-C₆alkynylthio, C₃-C₆cycloalkyl-C₁-C₆alkylthio, C₃-C₆haloalkynyl, C₂-C₆haloalkenylthio, C₁-C₆haloalkylthio, C₁-C₆alkoxy-C₁-C₆alkyl, C₁-C₆haloalkoxy-C₁-C₆alkyl, C₂-C₆alkenyloxy-C₁-C₆alkyl, C₂-C₆haloalkenyloxy-C₁-C₆alkyl, C₃-C₆alkynyloxy-C₁-C₆alkyl, NH₂, NHC₂-C₆alkenyl, C₂-C₆haloalkenyl, C₃-C₆-alkynyl, C₃-C₈cycloalkyl, C₃-C₈cycloalkyl-C₁-C₆alkyl, C₁-C₆alkoxy, C₁-C₆haloalkoxy, C₂-C₆alkenyloxy, C₂-C₆haloalkenyloxy, C₃-C₆alkynyloxy, C₃-C₆haloalkynyloxy, C₃-C₈cycloalkyl-C₁-C₆alkoxy, C₁-C₆alkylcarbonyl, C₁-C₆haloalkyl-carbonyl, C₁-C₆ alkoxycarbonyl, C₁-C₆alkylcarbonyl-C₁-C₆-alkyl, C₁-C₆alkoxycarbonyl-C₁-C₆alkyl, C₁-C₆alkylthio, C₂-C₆alkenylthio, C₃-C₆alkynylthio, C₃-C₆cycloalkyl-C₁-C₆alkylthio, C₃-C₆haloalkynyl, C₂-C₆haloalkenylthio, C₁-C₆haloalkylthio, C₁-C₆alkoxy-C₁-C₆alkyl, C₁-C₆haloalkoxy-C₁-C₆alkyl, C₂-C₆alkenyloxy-C₁-C₆alkyl, C₂-C₆haloalkenyloxy-C₁-C₆alkyl, C₃-C₆alkynyloxy-C₁-C₆alkyl, N(C₁-C₆alkyl)₂, the two alkyl groups independently of one another being able to be C₁-C₆alkylcarbonylamino, C₁-C₆haloalkylcarbonylamino, C₁-C₆alkoxycarbonylamino and C₁-C₆alkylaminocarbonylamino,
W is *C(R³²R³³) or *C(R³²R³³)C(R³⁴R³⁵), where the asterisk “*” denotes the attachment to oxygen, and in which R³² to R³⁵ each independently of one another are halogen, C₁-C₄alkyl, C₁-C₄alkoxy(C₁-C₄)alkyl or halo(C₁-C₄)alkyl,
n can adopt values from 0 to 3,
R⁸ is hydrogen, halogen, halo(C₁-C₄)alkyl, C₃-C₆cycloalkyl, C₂-C₅alkenyl, C₂-C₅alkynyl, C₁-C₄haloalkyl, C₁-C₄alkoxy, C₁-C₄haloalkoxy, C₁-C₄alkylthio, C₁-C₄haloalkylthio, C₁-C₄alkylsulfonyl, C₁-C₄haloalkylsulfonyl, nitro, cyano, aryl, alkylcarbonylamino, arylcarbonylamino and C₁-C₄alkoxycarbonylamino,
R¹⁰ and R¹¹ are in each case independently of one another hydrogen, halogen, hydroxyl, (C₁-C₄) alkyl, halo(C₁-C₄)alkyl or (C₁-C₄)alkoxy or together are carbonyl, —O—CH₂—CH₂—O—, S—CH₂—CH₂—S, ketal, thioketal or NOR², where
R¹² is (C₁-C₃)alkyl, (C₁-C₃)alkoxy(C₁-C₃)alkyl, aryl-(C₁-C₃)alkyl, (C₂-C₄)alkenyl-(C₁-C₃)alkyl, halo(C₂-C₄)alkenyl-(C₁-C₃)alkyl, di(C₁-C₃)alkylphosphonate, formyl, (C₁-C₃)alkylcarbonyl, halo(C₁-C₃)alkylcarbonyl, (C₁-C₃)alkoxy(C₁-C₃)alkylcarbonyl, arylcarbonyl and (C₁-C₃)alkylsulfonyl.

2. The compound of formula (I) according to claim 1, characterized in that
A¹ is one of the moieties —CH₂—CH═CCl₂, —CH₂—CH═CBr₂, —CH₂—CH═CClF, —CH₂—CH═CBrCl, —CH₂—CH═CBrF,
A²-X is a straight-chain or branched alkanediyl or alkenediyl having in each case up to 8 carbon atoms, which in each case may optionally be interrupted within the carbon chain by an oxygen atom, sulfur atom or a moiety selected from SO, SO₂, NH and N(C₁-C₄-alkyl), or
A²-X is —(C₁-C₄-alkyl)-(CO)—O—, —(C₁-C₄-alkyl)-(CO)—(NH)—, —(C₁-C₄-alkyl)-(CO)-[N—(C₁-C₄-alkyl)]-, —(C₁-C₄-alkyl)-(CO)—S—, (C₂-C₈-alkenyl)-(CO)—O—, —(C₂-C₈-alkenyl)-(CO)—(NH)—, —(C₂-C₈-alkenyl)-(CO)—[N—(C₁-C₄-alkyl)]-, —(C₂-C₈-alkenyl)-(CO)—S, piperidyl or piperazinyl, —(C₁-C₄-alkyl)-O—N═C(R⁹)—, —(C₁-C₆-alkenyl)-O—N═C(R⁹)—, where R⁹ either is methyl or is aryl, and there being located between X and R⁶, optionally, the bridging element 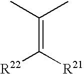
in which the radicals R²¹ and R²² may independently of one another be hydrogen, nitro, hydroxyl, amino, cyano, cyanato, thiocyanato, formyl, halogen or methyl,
R¹ is hydrogen, nitro, hydroxyl, cyano, fluoro, chloro, bromo, methyl, ethyl, n- or isopropyl, methoxy, ethoxy, n- or isopropoxy, methylthio, ethylthio, n- or isopropylthio, methylamino, ethylamino, n- or isopropylamino, dimethylamino or is the moiety —O-A¹, where A¹ has one of the definitions indicated above,
R² is hydrogen, nitro, cyano, cyanato, thiocyanato, formyl or halogen, is in each case optionally cyano-, halogen- or C₁-C₅-alkoxy-substituted alkyl, alkoxy, alkylthio, alkylamino, dialkylamino or alkylcarbonylamino having in each case 1 to 5 carbon atoms in the alkyl groups, is C₁-C₅-alkyl-carbonyl, C₁-C₅-alkoxy-carbonyl, C₁-C₅-alkoximinoformyl, C₁-C₅-alkoximino-acetyl, or is C₂-C₅-alkenyl or C₂-C₅-alkynyl,
R³ is hydrogen, nitro, halogen, is in each case optionally cyano-, halogen- or C₁-C₅-alkoxy-substituted alkyl, alkoxy, alkylthio or alkylamino having in each case 1 to 5 carbon atoms in the alkyl groups,
R⁴ is hydrogen, nitro, halogen, is in each case optionally cyano-, halogen- or C₁-C₅-alkoxy-substituted alkyl, alkoxy, alkylthio or alkylamino having in each case 1 to 5 carbon atoms in the alkyl groups,
R⁵ is an alkanyl moiety, an alkynyl moiety having in each case 2 to 5 carbon atoms or a cycloalkanyl moiety having 4 to 6 carbon atoms, which contains at least one substituent from the group nitro, cyano, carboxyl, carbamoyl, hydroxyl, carbonyl (C═O), hydroximino (C═N—OH), C₁-C₅-alkoxy, C₁-C₅-alkoxy-carbonyl, C₁-C₅-alkylamino, di(C₁-C₄-alkyl)-amino, C₁-C₅-alkylamino-carbonyl, C₁-C₅-alkoxy-carbonylamino, C₁-C₅-alkoxy-C₁-C₅-alkoxy, C₁-C₅-alkoxyimino, C₃-C₅-alkenyloxy, C₃-C₅-alkynyloxy, C₃-C₅-alkenyloxy-carbonyl, C₃-C₅-alkynyloxy-carbonyl, C₃-C₅-alkenyloxyimino, C₃-C₅-alkynyloxyimino, C₃-C₆-cycloalkyl,
R⁶ is an alkenyl moiety, an alkynyl moiety having in each case 2 to 6 carbon atoms or a cycloalkenyl moiety having 4 to 6 carbon atoms, which is optionally substituted by at least one substituent from the group nitro, cyano, carboxyl, carbamoyl, hydroxyl, carbonyl (C═O), hydroximino (C═N—OH), C₁-C₄-alkoxy, C₁-C₄-alkoxy-carbonyl, C₁-C₄-alkylamino, di(C₁-C₄-alkyl)-amino, C₁-C₄-alkylamino-carbonyl, C₁-C₄-alkoxy-carbonylamino, C₁-C₄-alkoxy-C₁-C₆-alkoxy, C₁-C₄-alkoxyimino, C₃-C₄-alkenyloxy, C₃-C₄-alkynyloxy, C₃-C₄-alkenyloxy-carbonyl, C₃-C₄-alkynyloxy-carbonyl, C₃-C₄-alkenyloxyimino, C₃-C₄-alkynyloxyimino, C₃-C₆-cycloalkyl, furyl, benzofuryl, thienyl, benzothienyl, isoxazolyl, benzisoxazolyl, pyrazolyl, pyridinyl, pyrimidinyl, pyrazinyl, pyridazinyl, or
R⁶ is a moiety -A³-Z, where
A³ is preferably a single bond or is straight-chain or branched and optionally halogen- or C₃-C₆-cycloalkyl-substituted alkanediyl having 1 to 6 carbon atoms, and
Z is in each case optionally nitro-, hydroxyl-, amino-, formyl-, cyano-, carboxyl-, carbamoyl-, fluoro-, chloro-, bromo-, methyl-, ethyl-, n- or isopropyl-, n-, iso-, s- or t-butyl-, fluoromethyl-, chloromethyl-, difluoromethyl-, dichloromethyl-, trifluoromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluoropropyl-, chloropropyl-, difluoropropyl-, dichloropropyl-, trifluoropropyl-, fluoroisopropyl-, difluoroisopropyl-, trifluoroisopropyl-, tetrafluoroisopropyl-, pentafluoroisopropyl-, methoxy-, ethoxy-, n- or isopropoxy-, fluoromethoxy-, difluoromethoxy-, trifluoromethoxy-, fluorodichloromethoxy-, chlorodifluoromethoxy-, fluoroethoxy-, chloroethoxy-, difluoroethoxy-, dichloroethoxy-, trifluoroethoxy-, fluoropropoxy, methoxycarbonyl-, ethoxycarbonyl-, fluoroethoxycarbonyl-, chloroethoxycarbonyl-, methoxymethyl-, ethoxymethyl-, n- or isopropoxymethyl-, methoxyethyl-, ethoxyethyl-, n- or isopropoxyethyl-, ethenyl-, propenyl-, butenyl-, fluoroethenyl-, chloroethenyl-, difluoroethenyl-, dichloroethenyl-, trifluoroethenyl-, trichloroethenyl-, propenyloxymethyl-, butenyloxymethyl-, propenyloxyethyl-, butenyloxyethyl-, ethynyl-, propynyl-, butynyl-, propenyloxy-, butenyloxy-, fluoropropenyloxy-, chloropropenyloxy-, fluorobutenyloxy-, chlorobutenyloxy-, propenyloxycarbonyl-, butenyloxycarbonyl-, propynyloxy-, butynyloxy-, propynyloxycarbonyl-, butynyloxycarbonyl-, propynyloxymethyl-, butynyloxymethyl-, propynyloxyethyl-, butynyloxyethyl-, cyclopropyl-, cyclobutyl-, cyclopentyl-, cyclopropylcarbonyl-, cyclobutylcarbonyl-, cyclopentylcarbonyl-, cyclopropylmethyl-, cyclobutylmethyl-, cyclopentylmethyl-, cyclohexylmethyl-, cyclopropylmethylcarbonyl-, cyclobutylmethylcarbonyl-, cyclopentylmethylcarbonyl-, cyclopropyloxy-, cyclobutyloxy-, cyclopentyloxy-, cyclohexyloxy-, cyclopropyloxycarbonyl-, cyclobutyloxycarbonyl-, cyclopentyloxycarbonyl-, cyclohexyloxycarbonyl-, cyclopropylmethoxy-, cyclobutylmethoxy-, cyclopentylmethoxy-, cyclohexylmethoxy-, cyclopropyl-methoxycarbonyl-, cyclobutylmethoxycarbonyl-, cyclopentylmethoxycarbonyl-, cyclohexylmethoxycarbonyl-, cyclopropylmethoxymethyl-, cyclobutylmethoxy-methyl-, cyclopentylmethoxymethyl-, cyclopropyloxymethoxy-, cyclobutyloxymethoxy-, cyclopentyloxymethoxy-, acetylmethyl-, propionylmethyl-, n- or isobutyroylmethyl-, acetylethyl-, propionylethyl-, methoxycarbonylmethyl-, ethoxycarbonylmethyl-, n- or isopropoxycarbonylmethyl-, methoxycarb-onylethyl-, ethoxycarbonylethyl-, n- or isopropoxycarbonylethyl-, methylthio-, ethylthio-, n- or isopropylthio-, difluoromethylthio-, trifluoromethylthio-, chloro-difluoromethylthio-, methylsulfinyl-, ethylsulfinyl-, trifluoromethylsulfinyl-, methylsulfonyl-, ethylsulfonyl-, trifluoromethylsulfonyl-, propenylthio-, butenylthio-, fluoropropenylthio-, chloropropenylthio-, fluorobutenylthio-, chlorobutenylthio-, propynylthio-, butynylthio-, cyclopropylthio-, cyclobutylthio-, cyclopentylthio-, cyclohexylthio-, cyclopropylmethylthio-, cyclobutylmethylthio-, cyclopentylmethylthio-, methylamino-, ethylamino-, n- or isopropylamino-, methylaminocarbonyl-, ethylaminocarbonyl-, n- or isopropyl-aminocarbonyl-, dimethylamino-, diethylamino-, dimethylaminocarbonyl-, diethylaminocarbonyl-, acetylamino-, propionylamino-, n- or isobutyroylamino-, methoxycarbonylamino-, ethoxycarbonylamino-, n- or isopropoxycarbonylamino-, methylaminocarbonyl-amino-, ethyl-aminocarbonylamino-, n- or isopropylaminocarbonylamino-substituted, or phenyl-, phenyloxy-, benzyl-, benzyloxy-, phenylamino- or benzylamino- (where in each case the phenyl groups are optionally substituted by nitro, hydroxyl, mercapto, amino, cyano, methyl, ethyl, n- or isopropyl, n-, iso-, s- or t-butyl, trifluoromethyl, methoxy, ethoxy, n- or isopropoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulfinyl, ethylsulfinyl, methylsulfonyl, ethylsulfonyl, methoxycarbonyl, ethoxy-carbonyl, n- or isopropoxycarbonyl) monocyclic heteroaryl having up to 5 carbon atoms and at least one heteroatom from the group N (nitrogen, 1 to 4 N atoms), O (oxygen, 1 O atom), sulfur (1 S atom) and optionally, in replacement or in addition, an SO or SO₂ moiety, and optionally in addition a carbonyl moiety (C═O) and/or a thiocarbonyl moiety (C═S) as part of the heterocycle, or
R⁶ is one of the moieties 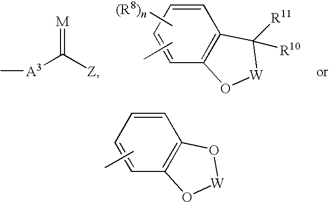
in which
M is oxygen or NOR⁷,
R⁷ is hydrogen; C₁-C₆alkyl, C₃-C₆cycloalkyl, C₁-C₄alkylcarbonyl, C₂-C₄alkenyl, C₂-C₄alkynyl, aryl, heterocyclyl or benzyl, where alkyl, cycloalkyl, alkenyl and alkynyl radicals are unsubstituted or are optionally substituted one to five times by a radical from the following group: halogen, —N₃, CN, NO₂, OH, SH, C₁-C₄alkoxy, C₁-C₄haloalkoxy, C₂-C₄alkenyloxy, C₂-C₄haloalkenyloxy, C₃-C₄alkynyloxy, C₃-C₄haloalkynyloxy, C₃-C₆cycloalkyl-C₁-C₄alkoxy, C₁-C₄alkylcarbonyl, C₁-C₄haloalkylcarbonyl, C₁-C₄alkoxycarbonyl, C₁-C₄alkylcarbonyl-C₁-C₄alkyl, C₁-C₄alkoxycarbonyl-C₁-C₄alkyl, C₁-C₄alkylthio, C₂-C₄alkylthio, C₃-C₄alkynylthio, C₃-C₄cycloalkyl-C₁-C₄alkylthio, C₃-C₄haloalkynyl, C₂-C₄haloalkenylthio, C₁-C₄haloalkylthio, C₁-C₄alkoxy-C₁-C₄alkyl, C₁-C₄haloalkoxy-C₁-C₄alkyl, C₂-C₄alkenyloxy-C₁-C₄alkyl, C₂-C₄haloalkenyloxy-C₁-C₄alkyl, C₃-C₄alkynyloxy-C₁-C₄alkyl, NH₂, NHC₂-C₄alkenyl, C₂-C₄haloalkenyl, C₃-C₆alkynyl, C₃-C₆cycloalkyl, C₃-C₆cycloalkyl-C₁-C₄alkyl, C₁-C₄alkoxy, C₁-C₄haloalkoxy, C₂-C₄alkenyloxy, C₂-C₄haloalkenyloxy, C₃-C₄alkynyloxy, C₃-C₄haloalkynyloxy, C₃-C₆cycloalkyl-C₁-C₄alkoxy, C₁-C₄alkylcarbonyl, C₁-C₄haloalkyl-carbonyl, C₁-C₄alkoxycarbonyl, C₁-C₄alkylcarbonyl-C₁-C₄alkyl, C₁-C₄alkoxycarbonyl-C₁-C₄alkyl, C₁-C₄alkylthio, C₂-C₄alkenylthio, C₃-C₄alkynylthio, C₃-C₆cycloalkyl-C₁-C₄alkylthio, C₃-C₄haloalkynyl, C₂-C₄haloalkenylthio, C₁-C₄haloalkylthio, C₁-C₄alkoxy-C₁-C₆alkyl, C₁-C₄haloalkoxy-C₁-C₄alkyl, C₂-C₄alkenyloxy-C₁-C₄alkyl, C₂-C₄haloalkenyloxy-C₁-C₄alkyl, C₃-C₄alkynyloxy-C₁-C₄alkyl, N(C₁-C₄alkyl)₂, where the two alkyl groups each independently of one another may be C₁-C₄alkylcarbonylamino, C₁-C₄haloalkylcarbonylamino, C₁-C₄-alkoxycarbonylamino and C₁-C₄alkylaminocarbonylamino,
R⁸ is hydrogen, halogen, halo(C₁-C₄)alkyl or nitro,
n is from 0 to 2,
R¹⁰ and R¹¹ taken together are ═O or alone are hydrogen and
W is C(R₃₂R₃₃), where R₃₂R₃₃ independently at each occurrence is halogen or C₁-C₄alkyl.

3. The compound of formula (I) according to claim 1, characterized in that
A¹ is one of the following moieties: —CH₂—CH═CCl₂, —CH₂—CH═CBr₂, —CH₂—CH═CBrCl,
A²-X is a straight-chain or branched alkanediyl or alkenediyl having in each case up to 8 carbon atoms, which may optionally be interrupted within the carbon chain by an oxygen atom, or
A²-X is —(C₁-C₄-alkyl)-(CO)—O—, —(C₁-C₄-alkyl)-(CO)—(NH)—, —(C₁-C₄-alkyl)-(CO)-[N—(C₁-C₄-alkyl)]-, —(C₁-C₄-alkyl)-(CO)—S—, (C₂-C₈-alkenyl)-(CO)—O—, —(C₂-C₈-alkenyl)-(CO)—(NH)—, —(C₂-C₈-alkenyl)-(CO)-[N—(C₁-C₄-alkyl)]-, —(C₂-C₈-alkenyl)-(CO)—S, piperidyl or piperazinyl, —(C₁-C₄-alkyl)-O—N═C(R⁹)—, —(C₁-C₆-alkenyl)-O—N═C(CH₃),
R⁹ is methyl or aryl, and
located between X and R⁶, optionally, is the bridging element 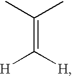
R¹ is hydrogen, nitro, hydroxyl, cyano, fluoro, chloro, bromo, methyl, ethyl, n- or isopropyl, methoxy, ethoxy, n- or isopropoxy, methylthio, ethylthio, n- or isopropylthio, methylamino, ethylamino, n- or isopropylamino or dimethylamino,
R² is hydrogen, nitro, cyano, cyanato, thiocyanato, formyl, fluoro, chloro, bromo, iodo, is in each case optionally cyano-, fluoro-, chloro-, methoxy-, ethoxy-, n- or isopropoxy-substituted methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino, dimethylamino, diethylamino, acetylamino, propionylamino, n- or isobutyroylamino, acetyl, propionyl, n- or isobutyroyl, methoxycarbonyl, ethoxycarbonyl, n- or isopropoxycarbonyl, methoximinoformyl, ethoximinoformyl, methoximinoacetyl or ethoximinoacetyl,
R³ is hydrogen, nitro, fluoro, chloro, bromo, iodo, is in each case optionally cyano-, fluoro-, chloro-, methoxy-, ethoxy-, n- or isopropoxy-substituted methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino,
R⁴ is hydrogen, nitro, fluoro, chloro, bromo, iodo, is in each case optionally cyano-, fluoro-, chloro-, methoxy-, ethoxy-, n- or isopropoxy-substituted methyl, ethyl, n- or isopropyl, n-, iso-, s- or t-butyl, methoxy, ethoxy, n- or isopropoxy, n-, iso-, s- or t-butoxy, methylthio, ethylthio, n- or isopropylthio, n-, iso-, s- or t-butylthio, methylamino, ethylamino, n- or isopropylamino, n-, iso-, s- or t-butylamino,
R⁵ is hydrogen, methyl, isopropyl, cyclopropyl or cyclohexyl,
R⁶ is one of moieties 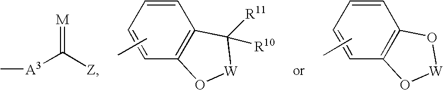
in which
A³ is a single bond or is in each case optionally fluoro-, chloro-, bromo-, cyclopropyl-, cyclobutyl-, cyclopentyl- or cyclohexyl-substituted methylene, ethane-1,1′-diyl (ethylidene), ethane-1,2-diyl (dimethylene), propane-1,1-diyl (propylidene), propane-1,2-diyl, propane-1,3-diyl (trimethylene), butane-1,1-diyl (butylidene) or butane-1,4-diyl (tetramethylene),
M is oxygen,
W is C(R₃₂R₃₃), where R₃₂R₃₃ independently at each occurrence is halogen or methyl,
Z is preferably in each case optionally nitro-, hydroxyl-, amino-, formyl-, cyano-, carboxyl-, carbamoyl-, fluoro-, chloro-, bromo-, methyl-, ethyl-, n- or isopropyl-, n-, iso-, s- or t-butyl-, fluoromethyl-, chloromethyl-, difluoromethyl-, dichloromethyl-, trifluoromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluoropropyl-, chloropropyl-, difluoropropyl-, dichloropropyl-, trifluoropropyl-, fluoroisopropyl-, difluoroisopropyl-, trifluoroisopropyl-, tetrafluoroisopropyl-, pentafluoroisopropyl-, methoxy-, ethoxy-, n- or isopropoxy-, fluoromethoxy-, difluoromethoxy-, trifluoromethoxy-, fluorodihloromethoxy-, chlorodifluoromethoxy-, fluoroethoxy-, chloroethoxy-, difluoroethoxy-, dichloroethoxy-, trifluoroethoxy-, fluoropropoxy, methoxycarbonyl-, ethoxycarbonyl-, fluoroethoxycarbonyl-, chloroethoxycarbonyl-, methoxymethyl-, ethoxymethyl-, n- or isopropoxymethyl-, methoxyethyl-, ethoxyethyl-, n- or isopropoxyethyl-, ethenyl-, propenyl-, butenyl-, fluoroethenyl-, chloroethenyl-, difluoroethenyl-, dichloroethenyl-, trifluoroethenyl-, trichloro-ethenyl-, propenyloxymethyl-, butenyloxymethyl-, propenyloxyethyl-, butenyloxyethyl-, ethynyl-, propynyl-, butynyl-, propenyloxy-, butenyloxy-, fluoropropenyloxy-, chloropropenyloxy-, fluorobutenyloxy-, chlorobutenyloxy-, propenyloxycarbonyl-, butenyloxycarbonyl-, propynyloxy-, butynyloxy-, propynyloxycarbonyl-, butynyloxycarbonyl-, propynyloxymethyl-, butynyloxymethyl-, propynyloxyethyl-, butynyloxyethyl-, cyclopropyl-, cyclobutyl-, cyclopentyl-, cyclopropylcarbonyl-, cyclobutylcarbonyl-, cyclopentylcarbonyl-, cyclopropylmethyl-, cyclobutylmethyl-, cyclopentylmethyl-, cyclohexylmethyl-, cyclopropylmethylcarbonyl-, cyclobutylmethylcarbonyl-, cyclopentylmethylcarbonyl-, cyclopropyloxy-, cyclobutyloxy-, cyclopentyloxy-, cyclohexyloxy-, cyclopropyloxycarbonyl-, cyclobutyloxycarbonyl-, cyclopentyloxycarbonyl-, cyclohexyloxycarbonyl-, cyclopropylmethoxy-, cyclobutylmethoxy-, cyclopentylmethoxy-, cyclohexyl-methoxy-, cyclopropylmethoxycarbonyl-, cyclobutylmethoxycarbonyl-, cyclopentylmethoxycarbonyl-, cyclohexylmethoxycarbonyl-, cyclopropylmethoxymethyl-, cyclobutylmethoxymethyl-, cyclopentylmethoxymethyl-, cyclopropyloxymethoxy-, cyclobutyloxymethoxy-, cyclopentyloxymethoxy-, acetylmethyl-, propionylmethyl-, n- or isobutyroylmethyl-, acetylethyl-, propionylethyl-, methoxycarbonylmethyl-, ethoxycarbonylmethyl-, n- or isopropoxycarbonylmethyl-, methoxycarb-onylethyl-, ethoxycarbonylethyl-, n- or isopropoxycarbonylethyl-, methylthio-, ethylthio-, n- or isopropylthio-, difluoromethylthio-, trifluoromethylthio-, chlorodifluoromethylthio-, methylsulfinyl-, ethylsulfinyl-, trifluoromethylsulfinyl-, methylsulfonyl-, ethylsulfonyl-, trifluoromethylsulfonyl-, propenylthio-, butenylthio-, fluoropropenylthio-, chloropropenylthio-, fluorobutenylthio-, chlorobutenylthio-, propynylthio-, butynylthio-, cyclopropylthio-, cyclobutylthio-, cyclopentylthio-, cyclohexylthio-, cyclopropylmethylthio-, cyclobutylmethylthio-, cyclopentylmethylthio-, methylamino-, ethylamino-, n- or isopropylamino-, methylaminocarbonyl-, ethylaminocarbonyl-, n- or isopropylaminocarbonyl-, dimethylamino-, diethylamino-, dimethylaminocarbonyl-, diethylaminocarbonyl-, acetylamino-, propionylamino-, n- or isobutyroylamino-, methoxycarbonylamino-, ethoxy-carbonylamino-, n- or isopropoxycarbonylamino-, methylaminocarbonylamino-, ethylaminocarbonylamino-, n- or isopropylaminocarbonylamino-substituted, or phenyl-, phenyloxy-, benzyl-, benzyloxy-, phenylamino- or benzylamino- (where in each case the phenyl groups are optionally substituted by nitro, hydroxyl, mercapto, amino, cyano, methyl, ethyl, n- or isopropyl, n-, iso-, s- or t-butyl, trifluoromethyl, methoxy, ethoxy, n- or isopropoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or isopropoxycarbonyl)pyrrolyl, pyrazolyl, imidazolyl, triazolyl, tetrazolyl, furyl, thienyl, oxazolyl, isoxazolyl, thiazolyl, isothiazolyl, oxadiazolyl, thiadiazolyl, pyridinyl, pyrimidinyl, pyridazinyl, pyrazinyl.

4. The compound of formula (I) according to claim 1, characterized in that
A¹ is the moiety —CH₂—CH═CCl₂,
A²-X is a straight-chain or branched alkanediyl having up to 8 carbon atoms, which optionally within the carbon chain contains an oxygen atom, and also is —(C₁-C₄-alkyl)-(CO)—O—, —(C₁-C₄-alkyl)-(CO)—(NH)— or piperidyl,
R¹ is hydrogen, methyl, ethyl, methoxy, ethoxy or fluoro, chloro, bromo,
R² is hydrogen, cyano, fluoro, chloro, bromo or iodo,
R² is hydrogen, fluoro, chloro or bromo,
R³ is hydrogen, cyano, fluoro, chloro, bromo, methyl, ethyl, methoxy or ethoxy,
R⁴ is hydrogen cyano, fluoro, chloro or bromo,
R⁵ is methyl,
R⁶ is a moiety 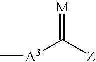
in which
A³ is a single bond or is methylene and
Z is preferably in each case optionally nitro-, hydroxyl-, amino-, formyl-, cyano-, carboxyl-, carbamoyl-, fluoro-, chloro-, bromo-, methyl-, ethyl-, n- or isopropyl-, n-, iso-, s- or t-butyl-, fluoromethyl-, chloromethyl-, difluoromethyl-, dichloromethyl-, trifluoromethyl-, trichloromethyl-, fluoroethyl-, chloroethyl-, difluoroethyl-, dichloroethyl-, chlorofluoroethyl-, trifluoroethyl-, trichloroethyl-, chlorodifluoroethyl-, fluoropropyl-, chloropropyl-, difluoropropyl-, dichloropropyl-, trifluoropropyl-, fluoroisopropyl-, difluoroisopropyl-, trifluoroisopropyl-, tetrafluoroisopropyl-, pentafluoroisopropyl-, methoxy-, ethoxy-, n- or isopropoxy-, fluoromethoxy-, difluoromethoxy-, trifluoromethoxy-, fluorodichloromethoxy-, chlorodifluoromethoxy-, fluoroethoxy-, chloroethoxy-, difluoroethoxy-, dichloroethoxy-, trifluoroethoxy-, fluoropropoxy, methoxycarbonyl-, ethoxycarbonyl-, fluoroethoxycarbonyl-, chloroethoxy-carbonyl-, methoxymethyl-, ethoxymethyl-, n- or isopropoxymethyl-, methoxyethyl-, ethoxyethyl-, n- or isopropoxyethyl-, ethenyl-, propenyl-, butenyl-, fluoroethenyl-, chloroethenyl-, difluoroethenyl-, dichloroethenyl-, trifluoroethenyl-, trichloro-ethenyl-, propenyloxymethyl-, butenyloxymethyl-, propenyloxyethyl-, butenyloxyethyl-, ethynyl-, propynyl-, butynyl-, propenyloxy-, butenyloxy-, fluoropropenyloxy-, chloropropenyloxy-, fluorobutenyloxy-, chlorobutenyloxy-, propenyloxycarbonyl-, butenyloxycarbonyl-, propynyloxy-, butynyloxy-, propynyloxycarbonyl-, butynyloxycarbonyl-, propynyloxymethyl-, butynyloxymethyl-, propynyloxyethyl-, butynyloxyethyl-, cyclopropyl-, cyclobutyl-, cyclopentyl-, cyclopropylcarbonyl-, cyclobutylcarbonyl-, cyclopentylcarbonyl-, cyclopropylmethyl-, cyclobutylmethyl-, cyclopentylmethyl-, cyclohexylmethyl-, cyclopropylmethylcarbonyl-, cyclobutylmethylcarbonyl-, cyclopentylmethylcarbonyl-, cyclopropyloxy-, cyclobutyloxy-, cyclopentyloxy-, cyclohexyloxy-, cyclopropyloxycarbonyl-, cyclobutyloxycarbonyl-, cyclopentyloxycarbonyl-, cyclohexyloxycarbonyl-, cyclopropylmethoxy-, cyclo-butylmethoxy-, cyclopentylmethoxy-, cyclohexylmethoxy-, cyclopropylmethoxycarbonyl-, cyclobutylmethoxycarbonyl-, cyclopentyl-methoxycarbonyl-, cyclohexylmethoxycarbonyl-, cyclopropylmethoxymethyl-, cyclobutylmethoxymethyl-, cyclopentylmethoxymethyl, cyclopropyloxymethoxy-, cyclobutyloxymethoxy-, cyclopentyloxymethoxy-, acetylmethyl-, propionylmethyl-, n- or isobutyroylmethyl-, acetylethyl-, propionylethyl-, methoxycarbonylmethyl-, ethoxycarbonylmethyl-, n- or isopropoxycarbonylmethyl-, methoxycarb-onylethyl-, ethoxycarbonylethyl-, n- or isopropoxycarbonylethyl-, methylthio-, ethylthio-, n- or isopropylthio-, difluoromethylthio-, trifluoromethylthio-, chlorodifluoromethylthio-, methylsulfinyl-, ethylsulfinyl-, trifluoromethylsulfinyl-, methylsulfonyl-, ethylsulfonyl-, trifluoromethylsulfonyl-, propenylthio-, butenylthio-, fluoropropenylthio-, chloropropenylthio-, fluorobutenylthio-, chlorobutenylthio-, propynylthio-, butynylthio-, cyclopropylthio-, cyclobutylthio-, cyclopentylthio-, cyclohexylthio-, cyclopropylmethylthio-, cyclobutylmethylthio-, cyclopentylmethylthio-, methylamino-, ethylamino-, n- or isopropylamino-, methylaminocarbonyl-, ethylaminocarbonyl-, n- or isopropylaminocarbonyl-, dimethylamino-, diethylamino-, dimethylaminocarbonyl-, diethylaminocarbonyl-, acetylamino-, propionylamino-, n- or isobutyroylamino-, methoxycarbonylamino-, ethoxy-carbonylamino-, n- or isopropoxycarbonylamino-, methylaminocarbonylamino-, ethylaminocarbonylamino-, n- or isopropylaminocarbonylamino-substituted, or phenyl-, phenyloxy-, benzyl-, benzyloxy-, phenylamino- or benzylamino- (where in each case the phenyl groups are optionally substituted by nitro, hydroxyl, mercapto, amino, cyano, methyl, ethyl, n- or isopropyl, n-, iso-, s- or t-butyl, trifluoromethyl, methoxy, ethoxy, n- or isopropoxy, difluoromethoxy, trifluoromethoxy, fluoroethoxy, difluoroethoxy, trifluoroethoxy, propenyloxy, butenyloxy, propynyloxy, butynyloxy, propynylthio, butynylthio, methylsulfinyl, ethylsulfinyl, methylsulfonyl, ethylsulfonyl, methoxycarbonyl, ethoxycarbonyl, n- or isopropoxycarbonyl) tetrazolyl.

5. A process for preparing a compound of formula (I) according to claim 1, comprising reacting a compound of formula (II), 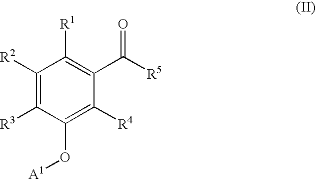
with a compound of formula H₂N—O-A²-X—R⁶, where the radicals R¹, R², R³, A¹, R⁴, R⁵, A², X and R⁶ have the definitions specified in claim 1, or by reaction of a compound of formula (IV) 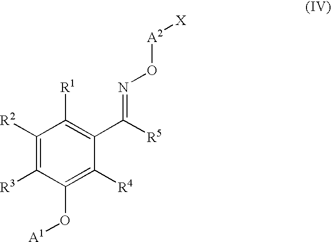
with a compound of a formula selected from: LG-R⁶, HO—R⁶, H₂N—R⁶ or LG-CO—R⁶, where the radicals R¹, R², R³, A¹, R⁴, R⁵, A², X and R⁶ have the definitions specified in claim 1 and LG is a leaving group.

6. A composition comprising at least one compound of the formula (I) according to claim 1 and at least one extender or at least one surface-active substance or at least one of both.

7. A method of controlling pests, comprising contacting a compound of formula (I) according to claim 1 or a composition according to claim 6 with the pests or their habitat or both.

8. (canceled)
